SEQUENCE LISTING (17 sequences)

SEQ ID NO: 1 - protein, 353 aa, synthetic construct
MAKIDNAVLPEGSLVLVTGANGFVGSHVVEQLLEHGYKVRGTARSASKLANLQKRWDAKYPGRFETAVVEDMLKQGAYDEVIKGAAGVAHIASVVSFSNKYDEVVTPAIGGTLNALRAAAATPSVKRFVLTSSTVSALIPKPNVEGIYLDEKSWNLESIDKAKTLPESDPQKSLWVYAASKTEAELAAWKFMDENKPHFTLNAVLPNYTIGTIFDPETQSGSTSGWMMSLFNGEVSPALALMPPQYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETAGQTGHHHHHH

SEQ ID NO: 2 - protein, 353 aa, synthetic construct
MKKIDNAVLPAGSLVLVTGANGFVGSHVVEQLLEHGYKVRGTARSASKLANLQKRWDAKYPGRFETAVVEDMLKDGAYDEVIKGAAGVAHIASPVSFSPKYDEVVPPAIGGTLNALRAAAATPSVKRFVLTSSMMAAIVPKPNVPGIYLDEKSWNLESIDKAVTLPESHKEKGLWVYAASKTMAEMLAWHFMDENKPHFTLNTVLPAYTIGTIFDPETQSGSTSGIVMKLFNGEVSPMLALFGPQHYVSAFDIGLLHLGCLVLPQIERRRVYGTAAPFDWNMVLAVFRKLWPSKTFPADFPDQGQDLSVFDTRPSLEILKSLGREGWRSFEDSIKDLVGSEFDGQTGHHHHHH

SEQ ID NO: 3 - DNA, 4203 bp, synthetic construct
  source 1..4203: mol_type = other DNA
tcgagttaattaaggcagtgagcgcaacgcaattaatgtgagttagctcactcattaggcaccccaggctttacactttatgcttccggctcgtatgttgtgtggaattgtgagcggataacaatttcacacaggaaacggctatgaccatgattacggattcactggccgtcgttttacaatctagaggccagcctggccataaggagatatacatatgaaaaagattgataacgcagttctgccggcagggtccctggttctggttaccggtgctaacggtttcgttggttcccacgtggttgaacagctgctggaacacggttacaaagttcgtggtaccgctcgttccgcttccaaactggctaacctgcagaaacgttgggatgctaaatacccgggtcgtttcgaaaccgctgttgtggaagacatgctgaaagatggtgcttacgacgaagttatcaaaggtgctgctggtgttgctcacatcgcttccccagtgagtttcagcccgaaatatgacgaagttgttcctccggctatcggtggtaccctgaacgctctgcgtgctgctgctgctaccccgtccgttaaacgtttcgttctgacctcctccatgatggcggctatcgtgccaaaaccgaacgttccgggtatctacctggacgaaaaatcctggaacctggaatccatcgacaaggctgtcacgctgccagaatcccacaaagaaaaaggtctgtgggtatacgctgcatccaagaccatggctgaaatgctggcatggcactttatggatgagaacaagccacacttcactctgaacaccgtactgccagcgtacactattggcactattttcgatccggaaactcagtcgggttccacctccggtattgttatgaaactgtttaacggcgaggtttccccgatgctggctctgtttgggccgcagcattacgtttccgctttcgatattggcctgctgcacctgggttgcctggttctgccacaaatcgaacgtcgtcgtgtttacggtacggctgctccgttcgattggaacatggttctggctgtcttccgtaaactgtggccgtccaaaaccttcccggctgacttcccagatcaaggtcaggacctgtctgtgtttgacacccgtccgtccctggaaattctgaaatctctgggtcgcgaaggttggcgttccttcgaagactccatcaaagacctggttggtagtgaatttgacggccaaactggccaccatcaccatcaccattagggaagagcagatgggcaagcttgacctgtgaagtgaaaaatggcgcacattgtgcgacatttttttttgaattctacgtaaaaagccgccgatacatcggctgctttttttttgatagaggttcaaacttgtggtataatgaaataagatcactccggggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataaacacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaagacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaactgcaggagctcaaacagcagcctgtattcaggctgcttttttcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgaaccaccgctggtagcggtggtttttttaggcctatggccttttttttttgtgggaaacctttcgcggtatggtattaaagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgtttccactgcgatgctggttgccaacgatcagatggcgctgggcgcaatgcgcgccattaccgagtccgggctgcgcgttggtgcggacatctcggtagtgggatacgacgataccgaagacagctcatgttatatcccgccgttaaccaccatcaaacaggattttcgcctgctggggcaaaccagcgtggaccgcttgctgcaactctctcagggccaggcggttaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcggtacccgataaaagcggcttcctgacaggaggccgttttgtttc

SEQ ID NO: 4 - protein, 353 aa, synthetic construct
MAKIDNAVLPEGSLVLVTGANGFVGSHVVEQLLEHGYKVRGTARSASKLANLQKRWDAKYPGRFETAVVEDMLKQGAYDEVIKGAAGVAHIASVVSFSNKYDEVVTPAIGGTLNALRAAAATPSVKRFVLTSSTVSALIPKPNVEGIYLDEKSWNLESIDKAKTLPESDPQKSLWVYAASKTEAELAAWKFMDENKPHFTLNAVNPNYTIGTIFDPETQSGSTSGWMMSLFNGEVSPALALVPPQYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETAGQTGHHHHHH

SEQ ID NO: 5 - protein, 353 aa, synthetic construct
MAKIDNAVLPEGSLVLVTGANGFVGSHVVEQLLEHGYKVRGTARSASKLANLQKRWDAKYPGRFETAVVEDMLKQGAYDEVIKGAAGVAHIASPVSFSNKYDEVVTPAIGGTLNALRAAAATPSVKRFVLTSSTMSALIPKPNVEGIYLDEKSWNLESIDKAKTLPESDPQKSLWVYAASKTEAELAAWKFMDENKPHFTLNAVNPNYTIGTIFDPETQSGSTSGWMMSLFNGEVSPALALVPPQYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETAGQTGHHHHHH

SEQ ID NO: 6 - protein, 353 aa, synthetic construct
MKKIDNAVLPEGSLVLVTGANGFVGSHVVEQLLEHGYKVRGTARSASKLANLQKRWDAKYPGRFETAVVEDMLKQGAYDEVIKGAAGVAHIASPVSFSNKYDEVVTPAIGGTLNALRAAAATPSVKRFVLTSSTMSALIPKPNVEGIYLDEKSWNLESIDKAKTLPESDPQKSLWVYAASKTEAELAAWKFMDENKPHFTLNAVNPNYTIGTIFDPETQSGSTSGWMMSLFNGEVSPALALVPPQYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETAGQTGHHHHHH

SEQ ID NO: 7 - protein, 353 aa, synthetic construct
MKKIDNAVLPAGSLVLVTGANGFVGSHVVEQLLEHGYKVRGTARSASKLANLQKRWDAKYPGRFETAVVEDMLKQGAYDEVIKGAAGVAHIASPVSFSNKYDEVVTPAIGGTLNALRAAAATPSVKRFVLTSSTMSALIPKPNVEGIYLDEKSWNLESIDKAKTLPESDPQKSLWVYAASKTEAELAAWKFMDENKPHFTLNAVNPNYTIGTIFDPETQSGSTSGWMMSLFNGEVSPALALFRPQYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETAGQTGHHHHHH

SEQ ID NO: 8 - protein, 353 aa, synthetic construct
MKKIDNAVLPAGSLVLVTGANGFVGSHVVEQLLEHGYKVRGTARSASKLANLQKRWDAKYPGRFETAVVEDMLKQGAYDEVIKGAAGVAHIASPVSFSNKYDEVVTPAIGGTLNALRAAAATPSVKRFVLTSSTMAALIPKPNVEGIYLDEKSWNLESIDKAKTLPESDPYKGLWVYAASKTEAELLAWKFMDENKPHFTLNAVNPNYTIGTIFDPETQSGSTSGWMMKLFNGEVSPALALFRPQYYVSAVDIGLLHLGCLVLPQIERRRVYGTAGTFDWNTVLATFRKLYPSKTFPADFPDQGQDLSKFDTAPSLEILKSLGRPGWRSFEESIKDLVGSETAGQTGHHHHHH

SEQ ID NO: 9 - protein, 353 aa, synthetic construct
MKKIDNAVLPAGSLVLVTGANGFVGSHVVEQLLEHGYKVRGTARSASKLANLQKRWDAKYPGRFETAVVEDMLKQGAYDEVIKGAAGVAHIASPVSFSNKYDEVVTPAIGGTLNALRAAAATPSVKRFVLTSSTMAALVPKPNVEGIYLDEKSWNLESIDKAKTLPESDPYKGLWVYAASKTTAELLAWKFMDENKPHFTLNAVAPAYTIGTIFDPETQSGSTSGFMMKLFNGEVSPALALFRPQYYVSAVDIGLLHLGCLVLPQIERRRVYGTAGTFDWNTVLATFRKLYPSKTFPADFPDQGQDLSKFDTAPSLEILKSLGRPGWRSFEESIKDLVGSETAGQTGHHHHHH

SEQ ID NO: 10 - protein, 353 aa, synthetic construct
MKKIDNAVLPAGSLVLVTGANGFVGSHVVEQLLEHGYKVRGTARSASKLANLQKRWDAKYPGRFETAVVEDMLKQGAYDEVIKGAAGVAHIASPVSFSNKYDEVVTPAIGGTLNALRAAAATPSVKRFVLTSSTMAALVPKPNVEGIYLDEKSWNLESIDKAKTLPESDPYKGLWVYAASKTTAELLAWKFMDENKPHFTLNAVAPAYTIGTIFDPETQSGSTSGFMMKLFNGEVSPMLALFAPQYYVSAVDIGLLHLGCLVLPQIERRRVYGTAGTFDWNGVLATFRKLWPSKTFPADFPDQGQDLSKFDTAPSLEILKSLGRPGWRSFEESIKDLVGSETAGQTGHHHHHH

SEQ ID NO: 11 - protein, 353 aa, synthetic construct
MKKIDNAVLPAGSLVLVTGANGFVGSHVVEQLLEHGYKVRGTARSASKLANLQKRWDAKYPGRFETAVVEDMLKQGAYDEVIKGAAGVAHIASPVSFSNKYDEVVTPAIGGTLNALRAAAATPSVKRFVLTSSTMAALVPKPNVEGIYLDEKSWNLESIDKAVTLPESDPEKGLWVYAASKTTAELLAWKFMDENKPHFTLNAVAPAYTIGTIFDPETQSGSTSGFMMKLFNGEVSPMLALFGPQYYVSAVDIGLLHLGCLVLPQIERRRVYGTAGTFDWNGVLATFRKLWPSKTFPADFPDQGQDLSKFDTAPSLEILKSLGRPGWRSFEDSIKDLVGSETAGQTGHHHHHH

SEQ ID NO: 12 - protein, 353 aa, synthetic construct
MKKIDNAVLPAGSLVLVTGANGFVGSHVVEQLLEHGYKVRGTARSASKLANLQKRWDAKYPGRFETAVVEDMLKQGAYDEVIKGAAGVAHIASPVLFSPKYDEVVTPAIGGTLNALRAAAATPSVKRFVLTSSTMAALVPKPNVEGIYLDEKSWNLESIDKAVTLPESDKEKGLWVYAASKTTAEMLAWKFMDENKPHFTLNTVLPAYTIGTIFDPETQSGSTSGFMMKLFNGEVSPMLALFGPQYYVSAVDIGLLHLGCLVLPQIERRRVYGTAGTFDWNGVLATFRKLWPSKTFPADFPDQGQDLSKFDTAPSLEILKSLGRPGWRSFEDSIKDLVGSETAGQTGHHHHHH

SEQ ID NO: 13 - protein, 353 aa, synthetic construct
MKKIDNAVLPAGSLVLVTGANGFVGSHVVEQLLEHGYKVRGTARSASKLANLQKRWDAKYPGRFETAVVEDMLKQGAYDEVIKGAAGVAHIASPVLFSPKYDEVVTPAIGGTLNALRAAAATPSVKRFVLTSSTMAALVPKPNVEGIYLDEKSWNLESIDKAVTLPESDKEKGLWVYAASKTTAEMLAWKFMDENKPHFTLNTVLPAYTIGTIFDPETQSGSTSGFVMKLFNGEVSPMLALFGPQHYVSAVDIGLLHLGCLVLPQIERRRVYGTAGTFDWNGVLATFRKLWPSKTFPADFPDQGQDLSKFDTTPSLEILKSLGRPGWRSFEDSIKDLVGSEFDGQTGHHHHHH

SEQ ID NO: 14 - protein, 353 aa, synthetic construct
MKKIDNAVLPAGSLVLVTGANGFVGSHVVEQLLEHGYKVRGTARSASKLANLQKRWDAKYPGRFETAVVEDMLKQGAYDEVIKGAAGVAHIASPVLFQPKYDEVVTPAIGGTLNALRAAAATPSVKRFVLTSSMMAAYVPKPNVQGIYLDEKSWNLESIDKAVTLPESDKEKGLWVYAASKTMAEMLAWKFMDENKPHFTLNTVLPAYTIGTIFDPETQSGSTSGIVMKLFNGEVSPMLALVGPQHYVSAVDIGLLHLGCLVLPQIERRRVYGTAGPFDWNMVLATFRKLWPSKTFPADFPDQGQDLSVFDTRPSLEILKSLGRPGWRSFEDSIKDLVGSEFDGQTGHHHHHH

SEQ ID NO: 15 - protein, 353 aa, synthetic construct
MKKIDNAVLPAGSLVLVTGANGFVGSHVVEQLLEHGYKVRGTARSASKLANLQKRWDAKYPGRFETAVVEDMLKQGAYDEVIKGAAGVAHIASPVSFSPKYDEVVTPAIGGTLNALRAAAATPSVKRFVLTSSMMAAYVPKPNVQGIYLDEKSWNLESIDKAPTLPESHKEKGLWVYAASKTMAEMLAWKFMDENKPHFTLNTVLPAYTIGTIFDPETQSGSTSGIVMKLFNGEVSPMLALFGPQHYVSAVDIGLLHLGCLVLPQIERRRVYGTAGPFDWNMVLATFRKLWPSKTFPADFPDQGQDLSVFDTRPSLEILKSLGREGWRSFEDSIKDLVGSEFDGQTGHHHHHH

SEQ ID NO: 16 - protein, 353 aa, synthetic construct
MKKIDNAVLPAGSLVLVTGANGFVGSHVVEQLLEHGYKVRGTARSASKLANLQKRWDAKYPGRFETAVVEDMLKDGAYDEVIKGAAGVAHIASPVSFSPKYDEVVPPAIGGTLNALRAAAATPSVKRFVLTSSMMAAIVPKPNVQGIYLDEKSWNLESIDKAVTLPESDKEKGLWVYAASKTMAEMLAWKFMDENKPHFTLNTVLPAYTIGTIFDPETQSGSTSGIVMKLFNGEVSPMLALFGPQHYVSAFDIGLLHLGCLVLPQIERRRVYGTAGPFDWNMVLATFRKLWPSKTFPADFPDQGQDLSVFDTRPSLEILKSLGRPGWRSFEDSIKDLVGSEFDGQTGHHHHHH

SEQ ID NO: 17 - DNA, 1062 bp, synthetic construct
  source 1..1062: mol_type = other DNA
atggctaaaatcgataacgcagttctgccggaaggttccctggttctggttaccggtgctaacggtttcgttggttcccacgttgttgaacagctgctggaacacggttacaaagttcgtggtaccgctcgttccgcttccaaactggctaacctgcagaaacgttgggacgctaaatacccgggtcgtttcgaaaccgctgttgttgaagacatgctgaaacagggtgcttacgacgaagttatcaaaggtgctgctggtgttgctcacatcgcttccgttgtttccttctccaacaaatacgacgaagttgttaccccggctatcggtggtaccttgaacgctctgcgtgctgctgctgctaccccgtccgttaaacgtttcgttctgacctcctccaccgtttccgctctgattccgaaaccgaacgttgaaggtatctacctggacgaaaaatcctggaacctggaatccatcgacaaagctaaaaccctgccggaatccgacccgcagaaatccctgtgggtatacgctgcatccaagaccgaagctgaactggctgcatggaaatttatggatgagaacaagccacacttcactctgaacgctgtactgccaaactacactattggcactattttcgatccggaaactcagtccggttccacctccggttggatgatgtccctgtttaacggcgaggtttccccggctctggctctgatgccaccgcagtactacgtttccgctgttgatattggcctgctgcacctgggttgcctggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctaaattcgacaccgctccgtccctggaaattctgaaatctctgggtcgcccaggttggcgttccatcgaagaatccatcaaagacctggttggttccgaaaccgctggccaaactggccaccatcaccatcaccattag

(19) United States
(12) Patent Application Publication
(10) Pub. No.: US 2026/0078354 A1
(43) Pub. Date: Mar. 19, 2026

(54) KETOREDUCTASE ENZYMES FOR THE SYNTHESIS OF 1,3-DIOL SUBSTITUTED INDANES

(71) Applicant: Merck Sharp & Dohme LLC, Rahway, NJ (US)

(72) Inventors: Jackson Kenai Blender Cahn, Clark, NJ (US); Wai Ling Cheung-Lee, Westfield, NJ (US); Stephanie W. Chun, Scotch Plains, NJ (US); Kaori Hiraga, North Bergen, NJ (US); Birgit Kosjek, Westfield, NJ (US); John Knight, Ocean View, NJ (US); Amanda M. Makarewicz, Gillette, NJ (US); Scott McCann, Middlesex, NJ (US); Jeffrey C. Moore, Westfield, NJ (US); Deeptak Verma, Feasterville-Trevose, PA (US)

(73) Assignee: Merck Sharp & Dohme LLC, Rahway, NJ (US)

(21) Appl. No.: 18/880,970

(22) PCT Filed: Jul. 5, 2023
(86) PCT No.: PCT/US2023/026889

§ 371 Date: Jan. 3, 2025

Related U.S. Application Data
(60) Provisional application No. 63/359,328, filed on Jul. 8, 2022.

(51) Int. Cl. C12N 9/04; C12N 15/70; C12P 11/00; C12R 1/19

(52) U.S. Cl. CPC C12N 9/0006; C12N 15/70; C12P 11/00; C12R 2001/19; C12Y 101/01

(57) ABSTRACT

The present disclosure provides ketoreductase enzymes having improved enzymatic properties including the capability of reducing hydroxy indanones to provide diastereomerically pure 1,3-indane diols useful for the synthesis of belzutifan. Also provided are polynucleotides encoding the ketoreductase enzymes, and host cells capable of expressing the ketoreductase enzymes. A purification procedure for isolating the ketoreductase enzymes is also provided.

### CROSS-REFERENCE TO RELATED APPLICATIONS

This application is a national stage of International Patent Application No. PCT/US2023/026889, filed Jul. 5, 2023, which claims the benefit of U.S. Provisional Patent Application No. 63/359,328, filed Jul. 8, 2022.

### REFERENCE TO SEQUENCE LISTING SUBMITTED ELECTRONICALLY

The instant application contains a Sequence Listing which has been submitted electronically in XML file format and is hereby incorporated by reference in its entirety. The XML file, created on Oct. 15, 2025, is named 25557-US-PCT_SL.xml and is 26,211 bytes in size.

### FIELD OF THE INVENTION

The present invention relates to ketoreductase enzymes, useful in biocatalytic and synthetic processes involving reduction of ketones to chiral alcohols. Such enzymes may be particularly useful in preparation of 1,3-diol substituted indanes.

### BACKGROUND OF THE INVENTION

Enzymes are polypeptides that serve to accelerate the chemical reactions of living cells (often by several orders of magnitude). Without enzymes, most biochemical reactions would be too slow to even carry out life processes. Enzymes display great specificity and are not permanently modified by their participation in reactions. Since they are not changed during the reactions, enzymes are particularly cost effective when used as catalysts for a desired chemical transformation.

Ketoreductases, also known as alcohol dehydrogenases, are enzymatic reducing agents, a specific class of enzymes that catalyze the selective reduction of ketones to chiral alcohols. Enzymes belonging to the ketoreductase or carbonyl reductase class are useful for the synthesis of optically active alcohols. Ketoreductase enzymes may selectively convert a ketone or aldehyde substrate to the corresponding chiral alcohol product; these enzymes may also convert alcohols into the corresponding ketones or aldehydes, in a reverse reaction. Enzymatic reduction of ketones and aldehydes requires participation of a co-factor that can act as an electron donor, while enzymatic oxidation of alcohols requires participation of a co-factor that can act as an electron acceptor.

Ketoreductase enzymes are well known in nature, and numerous genes that encode ketoreductase enzymes and ketoreductase enzyme sequences have been reported. See, e.g., Candida magnoliae (Genbank Acc. No. JC7338; GI:11360538) Candida parapsilosis (Genbank Acc. No. 10 BAA24528.1; GI:2815409), Sporobolomyces salmonicolor (Genbank Acc. No. AF160799; GI:6539734), and Rhodococcus erythropolis (Genbank Acc. No. AAN73270.1; GI: 34776951).

Ketoreductase enzymes are being used with increasing frequency to provide alternative synthetic pathways to key compounds. For example, Kosjek, B. et al. disclosed the asymmetric synthesis of a chiral precursor 4,4-dimethoxy-2H-pyran-3-ol with a ketoreductase. Organic Process Research & Development, 2008, 12. 584-588. When used, the ketoreductase enzymes may be provided as purified enzymes or as whole cells that express the desired ketoreductase. In view of their promise for improved synthetic pathways, there remains a need to identify additional ketoreductase enzymes that can be used to carry out certain chemical transformations to prepare specific chiral alcohols.

### SUMMARY OF THE INVENTION

The present disclosure relates to ketoreductase enzymes capable of converting ketones to chiral alcohols, particularly on a substituted indane scaffold. In some embodiments, the subject ketoreductase enzymes described herein are capable of converting hydroxy indanones to diastereomerically pure 1,3-indane diols useful for the synthesis of belzutifan, 3-[[(1S,2S,3R)-2,3-difluoro-2,3-dihydro-1-hydroxy-7-(methylsulfonyl)-1H-inden-4-yl]oxy]-5-fluorobenzonitrile. This agent is a hypoxia-inducible factor inhibitor and recently received approval from the Food and Drug Administration in the United States for the treatment of adult patients with von Hippel-Lindau disease who require therapy for associated renal cell carcinoma, central nervous system hemangioblastomas, or pancreatic neuroendocrine tumors, not requiring immediate surgery.

Additional embodiments describe processes for preparing the subject ketoreductase enzymes and processes for using the subject ketoreductase enzymes.

Other embodiments, aspects and features of the present invention are either further described in or will be apparent from the ensuing description, examples and appended claims.

### BRIEF DESCRIPTION OF THE DRAWINGS

FIG. 1 depicts SDS-PAGE gel shows the removal of iPrOH insoluble proteins from the lyophilized enzyme preparation.

### DETAILED DESCRIPTION OF THE INVENTION

### Definitions

Certain technical and scientific terms are specifically defined below. Unless specifically defined elsewhere in this document, all other technical and scientific terms used herein have the meaning commonly understood by one of ordinary skill in the art to which this disclosure relates. That notwithstanding and except where stated otherwise, the following definitions apply throughout the specification and claims. Chemical names, common names, and chemical structures may be used interchangeably to describe the same structure.

As used herein, and throughout this disclosure, the following terms, unless otherwise indicated, shall be understood to have the following meanings:

The present disclosure also embraces isotopically-labelled compounds that are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine and iodine, such as ²H, ³H, ¹¹C, ¹³C, ¹⁴C, ¹⁵N, 18O, ¹⁷O, ³¹P, ³²P, ³⁵S, ¹⁸F, ³⁶Cl, and ¹²³I, respectively.

Certain isotopically-labelled compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Isotopic substitution at a site where epimerization occurs may slow or reduce the epimerization process and thereby retain the more active or efficacious form of the compound for a longer period of time. Isotopically labeled compounds, in particular those containing isotopes with longer half-lives (T_{1/2}>1 day), can generally be prepared by following procedures analogous to those disclosed in the Schemes and/or in the Examples herein below, by substituting an appropriate isotopically labeled reagent for a non-isotopically labeled reagent.

Compounds herein may contain one or more stereogenic centers and can occur as racemates, racemic mixtures, single enantiomers, diastereomeric mixtures, and individual diastereomers. Additional asymmetric centers may be present depending upon the nature of the various substituents on the molecule. Each such asymmetric center will independently produce two optical isomers, and all possible optical isomers and diastereomers in mixtures and as pure or partially purified compounds are included within the disclosure. Any formulas, structures, or names of compounds described herein that do not specify a particular stereochemistry are meant to encompass any and all existing isomers as described above and mixtures thereof in any proportion. When stereochemistry is specified, the disclosure is meant to encompass that particular isomer in pure form or as part of a mixture with other isomers in any proportion.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Enantiomers can also be separated by use of chiral HPLC column.

All stereoisomers (for example, geometric isomers, optical isomers, and the like) of disclosed compounds (including those of the salts and solvates of compounds as well as the salts, solvates, and esters of prodrugs), such as those that may exist due to asymmetric carbons on various substituents, including enantiomeric forms (which may exist even in the absence of asymmetric carbons), rotameric forms, atropisomers, and diastereomeric forms, are contemplated within the scope of this disclosure. Individual stereoisomers of compounds may, for example, be substantially free of other isomers, or may be admixed, for example, as racemates or with all other, or other selected, stereoisomers. The chiral centers can have the S or R configuration as defined by the IUPAC 1974 Recommendations.

The present disclosure further includes compounds and synthetic intermediates in all their isolated forms. For example, the above-identified compounds are intended to encompass all forms of the compounds such as, any solvates, hydrates, stereoisomers, and tautomers thereof.

“Ketoreductase” and “KRED” are used interchangeably herein to refer to a polypeptide having an enzymatic capability of reducing a carbonyl group to its corresponding alcohol. More specifically, the ketoreductase polypeptides disclosed are capable of stereoselectively reducing the fluoro hydroxyindanone (6) (below) to the fluorodiol (7) (below).

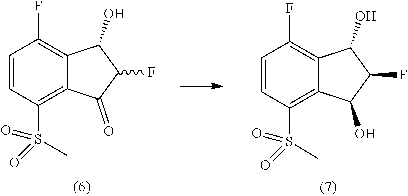 [SMILES: CS(=O)(=O)c1ccc(F)c2c1C(=O)C(F)[C@H]2O ; CS(=O)(=O)c1ccc(F)c2c1[C@H](O)[C@H](F)[C@H]2O]

The polypeptide typically utilizes a cofactor reduced nicotinamide adenine dinucleotide (NADH) or reduced nicotinamide adenine dinucleotide phosphate (NADPH) as the reducing agent. Ketoreductases as used herein include naturally occurring (wild type) ketoreductases as well as non-naturally occurring engineered polypeptides generated by human manipulation.

“Protein,” “polypeptide,” and “peptide” are used interchangeably herein to denote a polymer of at least two amino acids covalently linked by an amide bond, regardless of length or post-translational modification (e.g., glycosylation or phosphorylation, lipidation, myristoylation, ubiquitination, etc.). Included within this definition are D- and L-amino acids, and mixtures of D- and L-amino acids, as well as polymers comprising D- and L-amino acids, and mixtures of D- and L-amino acids. Proteins, polypeptides, and peptides may include a tag, such as a histidine tag, which should not be included when determining percentage of sequence identity.

“Amino acid” or “residue” as used in context of the polypeptides disclosed herein refers to the specific monomer at a sequence position. Amino acids are referred to herein by either their commonly known three-letter symbols or by the one-letter symbols recommended by IUPAC-IUB Biochemical Nomenclature Commission. Nucleotides, likewise, may be referred to by their commonly accepted single letter codes.

The abbreviations used for the genetically encoded amino acids are conventional and are as follows: alanine (Ala or A), arginine (Arg or R), asparagine (Asn or N), aspartate (Asp or D), cysteine (Cys or C), glutamate (Glu or E), glutamine (Gln or Q), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V).

The abbreviations used for the genetically encoding nucleosides are conventional and are as follows: adenosine (A); guanosine (G); cytidine (C); thymidine (T); and uridine (U). Unless specifically delineated, the abbreviated nucleosides may be either ribonucleosides or 2′-deoxyribonucleosides. The nucleosides may be specified as being either ribonucleosides or 2′-deoxyribonucleosides on an individual basis or on an aggregate basis. When nucleic acid sequences are presented as a string of one-letter abbreviations, the sequences are presented in the 5′ to 3′ direction in accordance with common convention, and the phosphates are not indicated.

“Derived from” as used herein in the context of enzymes, identifies the originating enzyme, and/or the gene encoding such enzyme, upon which the enzyme was based. For example, the ketoreductase enzyme of SEQ ID NO: 2 was obtained by artificially evolving, over multiple generations the gene encoding the ketoreductase enzyme of SEQ ID NO: 1. Thus, this evolved ketoreductase enzyme is “derived from” the ketoreductase of SEQ ID NO: 1.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides over a “comparison window” to identify and compare local regions of sequence similarity.

In some embodiments, a “reference sequence” can be based on a primary amino acid sequence, where the reference sequence is a sequence that can have one or more changes in the primary sequence. For instance, a reference sequence “based on SEQ ID NO:1 having at the residue corresponding to X190 a proline” refers to a reference sequence in which the corresponding residue at X190 in SEQ ID NO:1 has been changed to a proline.

“Hydrophilic amino acid or residue” refers to an amino acid or residue having a side chain exhibiting a hydrophobicity of less than zero according to the normalized consensus hydrophobicity scale of Eisenberg et al., 1984, J. Mol. Biol. 179:125-142. Genetically encoded hydrophilic amino acids include L-Thr (T), L-Ser (S), L-His (H), L-Glu (E), L-Asn (N), L-Gln (Q), L-Asp (D), L-Lys (K), and L-Arg (R).

“Acidic amino acid or residue” refers to a hydrophilic amino acid or residue having a side chain exhibiting a pK value of less than about 6 when the amino acid is included in a peptide or polypeptide. Acidic amino acids typically have negatively charged side chains at physiological pH due to loss of a hydrogen ion. Genetically encoded acidic amino acids include L-Glu (E) and L-Asp (D).

“Basic amino acid or residue” refers to a hydrophilic amino acid or residue having a side chain exhibiting a pKa value of greater than about 6 when the amino acid is included in a peptide or polypeptide. Basic amino acids typically have positively charged side chains at physiological pH due to association with hydronium ion. Genetically encoded basic amino acids include L-Arg (R) and L-Lys (K).

“Polar amino acid or residue” refers to a hydrophilic amino acid or residue having a side chain that is uncharged at physiological pH, but which has at least one bond in which the pair of electrons shared in common by two atoms is held more closely by one of the atoms. Genetically encoded polar amino acids include L-Asn (N), L-Gln (Q), L-Ser (S), and L-Thr (T).

“Hydrophobic amino acid or residue” refers to an amino acid or residue having a side chain exhibiting a hydrophobicity of greater than zero according to the normalized consensus hydrophobicity scale of Eisenberg et al., 1984, J. Mol. Biol. 179:125-142. Genetically encoded hydrophobic amino acids include L-Pro (P), L-Ile (I), L-Phe (F), L-Val (V), L-Leu (L), L-Trp (W), L-Met (M), L-Ala (A), and L-Tyr (Y).

“Aromatic amino acid or residue” refers to a hydrophilic or hydrophobic amino acid or residue having a side chain that includes at least one aromatic or heteroaromatic ring. Genetically encoded aromatic amino acids include L-Phe (F), L-Tyr (Y), L-His (H), and L-Trp (W). L-His (H) histidine is also classified herein as a hydrophilic residue or as a constrained residue.

As used herein, “constrained amino acid or residue” refers to an amino acid or residue that has a constrained geometry. Herein, constrained residues include L-Pro (P) and L-His (H). Histidine has a constrained geometry because it has a relatively small imidazole ring. Proline has a constrained geometry because it also has a five-membered ring.

“Non-polar amino acid or residue” refers to a hydrophobic amino acid or residue that has a side chain that is uncharged at physiological pH and that has bonds in which the pair of electrons shared in common by two atoms is generally held equally by each of the two atoms (i.e., the side chain is not polar). Genetically encoded non-polar amino acids include L-Gly (G), L-Leu (L), L-Val (V), L-Ile (I), L-Met (M), and L-Ala (A).

As used herein, “aliphatic amino acid or residue” refers to a hydrophobic amino acid or residue having an aliphatic hydrocarbon side chain. Genetically encoded aliphatic amino acids include L-Ala (A), L-Val (V), L-Leu (L), and L-Ile (I).

The ability of L-Cys (C) (and other amino acids with —SH containing side chains) to exist in a peptide in either the reduced free —SH or oxidized disulfide-bridged form affects whether L-Cys (C) contributes net hydrophobic or hydrophilic character to a peptide. While L-Cys (C) exhibits a hydrophobicity of 0.29 according to the normalized consensus scale of Eisenberg (Eisenberg et al., 1984, supra), it is to be understood that for purposes of the present disclosure, L-Cys (C) is categorized into its own unique group. It is noted that cysteine (or “L-Cys” or “[C]”) is unusual in that it can form disulfide bridges with other L-Cys (C) amino acids or other sulfanyl- or sulfhydryl-containing amino acids. The “cysteine-like residues” include cysteine and other amino acids that contain sulfhydryl moieties that are available for formation of disulfide bridges.

As used herein, “small amino acid or residue” refers to an amino acid or residue having a side chain that is composed of a total three or fewer carbon and/or heteroatoms (excluding the α-carbon and hydrogens). The small amino acids or residues may be further categorized as aliphatic, non-polar, polar or acidic small amino acids or residues, in accordance with the above definitions. Genetically-encoded small amino acids include L-Ala (A), L-Val (V), L-Cys (C), L-Asn (N), L-Ser (S), L-Thr (T), and L-Asp (D).

“Hydroxyl-containing amino acid or residue” refers to an amino acid containing a hydroxyl (—OH) moiety. Genetically-encoded hydroxyl-containing amino acids include L-Ser (S) L-Thr (T), and L-Tyr (Y).

As used herein, “conservative amino acid substitution” refers to a substitution of a residue with a different residue having a similar side chain, and thus typically involves substitution of the amino acid in the polypeptide with amino acids within the same or similar defined class of amino acids. By way of example and not limitation, in some embodiments, an amino acid with an aliphatic side chain is substituted with another aliphatic amino acid (e.g., alanine, valine, leucine, and isoleucine); an amino acid with an hydroxyl side chain is substituted with another amino acid with an hydroxyl side chain (e.g., serine and threonine); an amino acids having aromatic side chains is substituted with another amino acid having an aromatic side chain (e.g., phenylalanine, tyrosine, tryptophan, and histidine); an amino acid with a basic side chain is substituted with another amino acid with a basic side chain (e.g., lysine and arginine); an amino acid with an acidic side chain is substituted with another amino acid with an acidic side chain (e.g., aspartic acid and glutamic acid); and/or a hydrophobic or hydrophilic amino acid is replaced with another hydrophobic or hydrophilic amino acid, respectively.

As used herein, “non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

As used herein, “deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of an evolved enzyme. Deletions can be directed to the internal portions and/or terminal portions of the polypeptide. In various embodiments, the deletion can comprise a continuous segment or can be discontinuous. Deletions are typically indicated by “-” in amino acid sequences.

As used herein, “insertion” refers to modification to the polypeptide by addition of one or more amino acids from the reference polypeptide. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

The term “amino acid substitution set” or “substitution set” refers to a group of amino acid substitutions in a polypeptide sequence, as compared to a reference sequence. A substitution set can have 1, 2, 3, 4, 5, 6, 7, 8, 9, 10, 11, 12, 13, 14, 15, or more amino acid substitutions.

A “functional fragment” and “biologically active fragment” are used interchangeably herein to refer to a polypeptide that has an amino-terminal and/or carboxy-terminal deletion(s) and/or internal deletions, but where the remaining amino acid sequence is identical to the corresponding positions in the sequence to which it is being compared and that retains substantially all of the activity of the full-length polypeptide.

As used herein, “isolated polypeptide” refers to a polypeptide that is substantially separated from other contaminants that naturally accompany it (e.g., protein, lipids, and polynucleotides). The term embraces polypeptides that have been removed or purified from their naturally occurring environment or expression system (e.g., within a host cell or via in vitro synthesis). The recombinant polypeptides may be present within a cell, present in the cellular medium, or prepared in various forms, such as lysates or isolated preparations. As such, in some embodiments, the recombinant polypeptides can be an isolated polypeptide.

As used herein, “substantially pure polypeptide” or “purified protein” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. However, in some embodiments, an enzyme comprising composition comprises enzymes that are less than 50% pure (e.g., about 10%, about 20%, about 30%, about 40%, or about 50%). Generally, a substantially pure enzyme or polypeptide composition comprises about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated recombinant polypeptides are substantially pure polypeptide compositions.

“Improved enzyme property” refers to an enzyme that exhibits an improvement in any enzyme property as compared to a reference enzyme. For the enzymes described herein, the comparison is generally made to the wild-type enzyme, although in some embodiments, the reference enzyme can be another improved enzyme. Enzyme properties for which improvement is desirable include, but are not limited to, enzymatic activity (which can be expressed in terms of percent conversion of the substrate), thermal stability, pH activity profile, cofactor requirements, refractoriness to inhibitors (e.g., product inhibition), stereospecificity, and stereoselectivity (including enantioselectivity).

“Increased enzymatic activity” refers to an improved property of the enzymes, which can be represented by an increase in specific activity (e.g., product produced/time/weight protein) or an increase in percent conversion of the substrate to the product (e.g., percent conversion of starting amount of substrate to product in a specified time period using a specified amount of enzyme) as compared to the reference enzyme. Exemplary methods to determine enzyme activity are provided in the Examples. Any property relating to enzyme activity may be affected, including the classical enzyme properties of Kₘ, Vₘₐₓ, or k_{cat}, changes of which can lead to increased enzymatic activity. Improvements in enzyme activity can be from about 1.5 times the enzymatic activity of the corresponding wild-type enzyme, to as much as 2 times. 5 times, 10 times, 20 times, 25 times, 50 times, 75 times, 100 times, 150 times, 200 times, 500 times, 1000 times, 3000 times, 5000 times, 7000 times or more enzymatic activity than the naturally occurring enzyme or another enzyme from which the polypeptides were derived. In specific embodiments, the enzyme exhibits improved enzymatic activity in the range of 150 to 3000 times, 3000 to 7000 times, or more than 7000 times greater than that of the parent enzyme. It is understood by the skilled artisan that the activity of any enzyme is diffusion limited such that the catalytic turnover rate cannot exceed the diffusion rate of the substrate, including any required cofactors. The theoretical maximum of the diffusion limit, or k_{cat}/Kₘ, is generally about 10⁸ to 10⁹ (M⁻¹s⁻¹). Hence, any improvements in the enzyme activity will have an upper limit related to the diffusion rate of the substrates acted on by the enzyme. Enzyme activity can be measured by any one of standard assays used for measuring kinase activity, or via a coupled assay with an nucleoside phosphorylase enzyme which is capable of catalyzing reaction between the polypeptide product and a nucleoside base to afford a nucleoside, or by any of the traditional methods for assaying chemical reactions, including but not limited to HPLC, HPLC-MS, UPLC, UPLC-MS, TLC, and NMR. Comparisons of enzyme activities are made using a defined preparation of enzyme, a defined assay under a set condition, and one or more defined substrates, as further described in detail herein. Generally, when lysates are compared, the numbers of cells and the amount of protein assayed are determined as well as use of identical expression systems and identical host cells to minimize variations in amount of enzyme produced by the host cells and present in the lysates.

As used herein, a “vector” is a DNA construct for introducing a DNA sequence into a cell. In some embodiments, the vector is an expression vector that is operably linked to a suitable control sequence capable of effecting the expression in a suitable host of the polypeptide encoded in the DNA sequence. In some embodiments, an “expression vector” has a promoter sequence operably linked to the DNA sequence (e.g., transgene) to drive expression in a host cell, and in some embodiments, also comprises a transcription terminator sequence.

As used herein, the term “expression” includes any step involved in the production of the polypeptide including, but not limited to, transcription, post-transcriptional modification, translation, and post-translational modification. In some embodiments, the term also encompasses secretion of the polypeptide from a cell.

As used herein, the term “produces” refers to the production of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

As used herein, an amino acid or nucleotide sequence (e.g., a promoter sequence, signal peptide, terminator sequence, etc.) is “heterologous” to another sequence with which it is operably linked if the two sequences are not associated in nature. For example, a “heterologous polynucleotide” is any polynucleotide that is introduced into a host cell by laboratory techniques, and the term includes polynucleotides that are removed from a host cell, subjected to laboratory manipulation, and then reintroduced into a host cell.

As used herein, the terms “host cell” and “host strain” refer to suitable hosts for expression vectors comprising DNA provided herein (e.g., the polynucleotides encoding the variants). In some embodiments, the host cells are prokaryotic or eukaryotic cells that have been transformed or transfected with vectors constructed using recombinant DNA techniques as known in the art.

The term “analogue” means a polypeptide having more than 70% sequence identity but less than 100% sequence identity (e.g., more than 75%, 78%, 80%, 83%, 85%, 88%, 90%, 91%, 92%, 93%, 94%, 95%, 96%, 97%, 98%, or 99% sequence identity) with a reference polypeptide. In some embodiments, “analogues” means polypeptides that contain one or more non-naturally occurring amino acid residues including, but not limited, to homoarginine, ornithine and norvaline, as well as naturally occurring amino acids. In some embodiments, analogues also include one or more D-amino acid residues and non-peptide linkages between two or more amino acid residues.

As used herein, “EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

As used herein, “ATCC” refers to the American Type Culture Collection whose biorepository collection includes genes and strains.

As used herein, “NCBI” refers to National Center for Biological Information and the sequence databases provided therein.

“Coding sequence” refers to that portion of a nucleic acid (e.g., a gene) that encodes an amino acid sequence of a protein.

“Naturally occurring” or “wild-type” refers to a form found in nature. For example, a naturally occurring or wild-type polypeptide or polynucleotide sequence is a sequence present in an organism that can be isolated from a source in nature and that has not been intentionally modified by human manipulation, with the sole exception that wild-type polypeptide or polynucleotide sequences as identified herein may include a tag, such as a histidine tag, which should not be included when determining percentage of sequence identity. Herein, “wild-type” polypeptide or polynucleotide sequences may be denoted “WT”.

“Recombinant” when used with reference to, e.g., a cell, nucleic acid, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Percentage of sequence identity,” “percent identity,” and “percent identical” are used herein to refer to comparisons between polynucleotide sequences or polypeptide sequences, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage is calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Determination of optimal alignment and percent sequence identity is performed using the BLAST and BLAST 2.0 algorithms (see e.g., Altschul et al., 1990, J. Mol. Biol. 215: 403-410; and Altschul et al., 1977, Nucleic Acids Res. 3389-3402). Software for performing BLAST analyses is publicly available through the National Center for Biotechnology Information website.

Briefly, the BLAST analyses involve first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff and Henikoff, 1989, PROC. NATL. ACAD. SCI. USA 89:10915).

Numerous other algorithms are available that function similarly to BLAST in providing percent identity for two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Additionally, determination of sequence alignment and percent sequence identity can employ the BESTFIT or GAP programs in the GCG Wisconsin Software package (Accelrys, Madison WI), using default parameters provided.

“Substantial identity” refers to a polynucleotide or polypeptide sequence that has at least 80 percent sequence identity, preferably at least 85 percent sequence identity, more preferably at least 89 percent sequence identity, more preferably at least 95 percent sequence identity, and even more preferably at least 99 percent sequence identity as compared to a reference sequence over a comparison window of at least 20 residue positions, frequently over a window of at least 30-50 residues, wherein the percentage of sequence identity is calculated by comparing the reference sequence to a sequence that includes deletions or additions which total 20 percent or less of the reference sequence over the window of comparison. In specific embodiments applied to polypeptides, the term “substantial identity” means that two polypeptide sequences, when optimally aligned, such as by the programs GAP or BESTFIT using default gap weights, share at least 80 percent sequence identity, preferably at least 89 percent sequence identity, more preferably at least 95 percent sequence identity or more (e.g., 99 percent sequence identity). Preferably, residue positions which are not identical differ by conservative amino acid substitutions.

“Corresponding to”, “reference to” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Stereoselectivity” refers to the preferential formation in a chemical or enzymatic reaction of one stereoisomer over another. Stereoselectivity can be partial, where the formation of one stereoisomer is favored over the other, or it may be complete where only one stereoisomer is formed. When the stereoisomers are enantiomers, the stereoselectivity is referred to as enantioselectivity, the fraction (typically reported as a percentage) of one enantiomer in the sum of both. It is commonly alternatively reported in the art (typically as a percentage) as the enantiomeric excess (EE) calculated therefrom according to the formula [major enantiomer−minor enantiomer]/[major enantiomer+minor enantiomer]. Where the stereoisomers are diastereoisomers, the stereoselectivity is referred to as diastereoselectivity, the fraction (typically reported as a percentage) of one diastereomer in a mixture of two diastereomers, commonly alternatively reported as the diastereomeric excess (DE). Enantiomeric excess and diastereomeric excess are types of stereomeric excess.

“Highly stereoselective” refers to a chemical or enzymatic reaction that is capable of converting a substrate to its corresponding product with at least about 85% stereoisomeric excess.

“Chemoselectivity” refers to the preferential formation in a chemical or enzymatic reaction of one product over another.

“Conversion” refers to the enzymatic transformation of a substrate to the corresponding product. “Percent conversion” refers to the percent of the substrate that is converted to the product within a period of time under specified conditions. Thus, for example, the “enzymatic activity” or “activity” of a polypeptide can be expressed as “percent conversion” of the substrate to the product.

“Chiral alcohol” refers to amines of general formula R¹—CH(OH)—R² wherein R¹ and R² are nonidentical and is employed herein in its broadest sense, including a wide variety of aliphatic and alicyclic compounds of different, and mixed, functional types, characterized by the presence of a primary hydroxyl group bound to a secondary carbon atom which, in addition to a hydrogen atom, carries either (i) a divalent group forming a chiral cyclic structure, or (ii) two substituents (other than hydrogen) differing from each other in structure or chirality. Divalent groups forming a chiral cyclic structure include, for example, 2-methylbutane-1,4-diyl, pentane-1,4-diyl, hexane-1,4-diyl, hexane-1,5-diyl, 2-methylpentane-1,5-diyl. The two different substituents on the secondary carbon atom (R¹ and R² above) also can vary widely and include alkyl, aralkyl, aryl, halo, hydroxy, lower alkyl, lower alkoxy, lower alkylthio, cycloalkyl, carboxy, carboalkoxy, carbamoyl, mono- and di-(lower alkyl) substituted carbamoyl, trifluoromethyl, phenyl, nitro, amino, mono- and di-(lower alkyl) substituted amino, alkylsulfonyl, arylsulfonyl, alkylcarboxamido, arylcarboxamido, etc., as well as alkyl, aralkyl, or aryl substituted by the foregoing.

Immobilized enzyme preparations have a number of recognized advantages. They can confer shelf life to enzyme preparations, they can improve reaction stability, they can enable stability in organic solvents, they can aid in protein removal from reaction streams, as examples. “Stable” refers to the ability of the immobilized enzymes to retain their structural conformation and/or their activity in a solvent system that contains organic solvents. Stable immobilized enzymes lose less than 10% activity per hour in a solvent system that contains organic solvents. Stable immobilized enzymes lose less than 9% activity per hour in a solvent system that contains organic solvents. Preferably, the stable immobilized enzymes lose less than 8% activity per hour in a solvent system that contains organic solvents. Preferably, the stable immobilized enzymes lose less than 7% activity per hour in a solvent system that contains organic solvents. Preferably, the stable immobilized enzymes lose less than 6% activity per hour in a solvent system that contains organic solvents. Preferably, the stable immobilized enzymes lose less than 5% activity per hour in a solvent system that contains organic solvents. Preferably, the stable immobilized enzymes less than 4% activity per hour in a solvent system that contains organic solvents. Preferably, the stable immobilized enzymes lose less than 3% activity per hour in a solvent system that contains organic solvents. Preferably, the stable immobilized enzymes lose less than 2% activity per hour in a solvent system that contains organic solvents. Preferably, the stable immobilized enzymes lose less than 1% activity per hour in a solvent system that contains organic solvents.

“Thermostable” refers to a polypeptide that maintains similar activity (more than 60% to 80%, for example) after exposure to elevated temperatures (e.g., 40° C. to 80° C.) for a period of time (e.g., 0.5 h to 24 h) compared to the untreated enzyme.

“Solvent stable” refers to a polypeptide that maintains similar activity (more than e.g., 60% to 80%) after exposure to varying concentrations (e.g., 5% to 99%) of solvent (isopropyl alcohol, tetrahydrofuran, 2-methyltetrahydrofuran, acetone, toluene, butylacetate, methyl tert-butylether, etc.) for a period of time (e.g., 0.5 h to 24 h) compared to the untreated enzyme.

“pH stable” refers to a polypeptide that maintains similar activity (more than e.g., 60% to 80%) after exposure to high or low pH (e.g., 4.5 to 6 or 8 to 12) for a period of time (e.g., 0.5 h to 24 h) compared to the untreated enzyme.

“Thermo- and solvent stable” refers to a polypeptide that is both thermostable and solvent stable.

As used herein, the terms “biocatalysis,” “biocatalytic,” “biotransformation,” and “biosynthesis” refer to the use of enzymes to perform chemical reactions on organic compounds.

The term “effective amount” means an amount sufficient to produce the desired result. One of general skill in the art may determine what the effective amount by using routine experimentation.

The terms “isolated” and “purified” are used to refer to a molecule (e.g., an isolated nucleic acid, polypeptide, etc.) or other component that is removed from at least one other component with which it is naturally associated. The term “purified” does not require absolute purity, rather it is intended as a relative definition.

“Control sequence” is defined herein to include all components, which are necessary or advantageous for the expression of a polynucleotide and/or polypeptide of interest. Each control sequence may be native or foreign to the nucleic acid sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with a polynucleotide of interest, e.g., the coding region of the nucleic acid sequence encoding a polypeptide.

“Operably linked” is defined herein as a configuration in which a control sequence is appropriately placed at a position relative to a polynucleotide sequence (i.e., in a functional relationship) such that the control sequence directs the expression of the polynucleotide and/or a polypeptide encoded by the polynucleotide.

“Promoter sequence” is a nucleic acid sequence that is recognized by a host cell for expression of a polynucleotide. The control sequence may comprise an appropriate promoter sequence. The promoter sequence contains transcriptional control sequences, which mediate the expression of the polynucleotide. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

Exemplary methods and materials are described herein, although methods and materials similar or equivalent to those described herein can also be used in the practice or testing of the present disclosure. The materials, methods, and examples are illustrative only and not intended to be limiting.

### ABBREVIATIONS

ACN, MeCN Acetonitrile
DMSO Dimethyl sulfoxide
g Grams
μg microgram
h Hour
Hz Hertz
iPrOH Isopropyl alcohol
IPTG Isopropyl β-D-1-thiogalactopyranoside
J NMR Coupling constant
L Liter
M Molar, moles per liter
mg Milligrams
min Minute(s)
mL, ml Milliliters
mm Millimeter
mM Millimole per liter
m micrometer
nm Nanometer
NaCl Sodium chloride
NADP Nicotinamide adenine dinucleotide phosphate
OD₆₀₀ Optical density at 600 nm wavelength
PTFE Polytetrafluoroethylene
RH Relative humidity
RPM, rpm Revolutions per minute
RT Room temperature, approximately 25° C.
TFA Trifluoroacetic acid
THF Tetrahydrofuran
UPLC Ultra-Performance Liquid Chromatography
vol Volumes
v/v Volume per volume
μg, ug Micrograms
μL, μl, uL, ul Microliters

### Ketoreductases

This disclosure relates to ketoreductase enzymes capable of reducing ketones to chiral alcohols particularly on a substituted indane scaffold. In embodiments, the ketoreductase enzymes are capable of the following conversion:

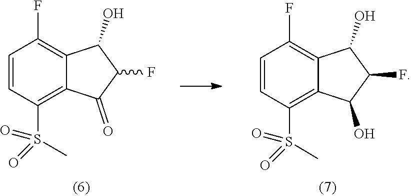 [SMILES: CS(=O)(=O)c1ccc(F)c2c1C(=O)C(F)[C@H]2O ; CS(=O)(=O)c1ccc(F)c2c1[C@H](O)[C@H](F)[C@H]2O]

In particular embodiments, the ketoreductase enzymes are utilized in conjunction with electrophilic fluorinating agents in the transformation:

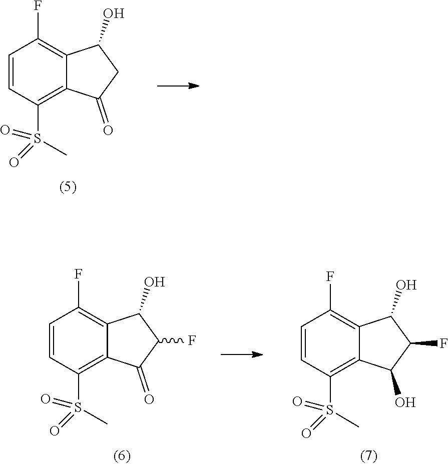 [SMILES: CS(=O)(=O)c1ccc(F)c2c1C(=O)C[C@H]2O ; CS(=O)(=O)c1ccc(F)c2c1C(=O)C(F)[C@H]2O ; CS(=O)(=O)c1ccc(F)c2c1[C@H](O)[C@H](F)[C@H]2O]

In embodiments, the ketoreductase enzymes described herein have an amino acid sequence that has one or more amino acid differences as compared to a reference amino acid sequence of a commercially available, non-wild type ketoreductase that result in an improved property of the enzyme for the defined keto substrate.

The ketoreductase enzymes described herein are the product of directed evolution from a commercially available ketoreductase (SEQ ID NO:1, Codexis, Inc.), which was identified by screening a collection of Codexis enzymes. and which has the amino acid sequence as set forth below:

(SEQ ID NO: 1)
MAKIDNAVLPEGSLVLVTGANGFVGSHVVEQLLEHGYKVRGTARSASKL
ANLQKRWDAKYPGRFETAVVEDMLKQGAYDEVIKGAAGVAHIASVVSFS
NKYDEVVTPAIGGTLNALRAAAATPSVKRFVLTSSTVSALIPKPNVEGI
YLDEKSWNLESIDKAKTLPESDPQKSLWVYAASKTEAELAAWKFMDENK
PHFTLNAVLPNYTIGTIFDPETQSGSTSGWMMSLFNGEVSPALALMPPQ
YYVSAVDIGLLHLGCLVLPQIERRRVYGTAGTFDWNTVLATFRKLYPSK
TFPADFPDQGQDLSKFDTAPSLEILKSLGRPGWRSIEESIKDLVGSETA
GQTGHHHHHH.

In embodiments, the ketoreductase enzymes of the disclosure may demonstrate improvements relative the ketoreductase enzyme of SEQ ID NO: 1, such as increases in enzyme activity, stereoselectivity, stereospecificity, thermostability, solvent stability, or reduced product inhibition.

In some embodiments, the ketoreductase enzymes of the disclosure may demonstrate improvements in the rate of enzymatic activity, i.e., the rate of converting the substrate to the product. In some embodiments, the ketoreductase polypeptides are capable of converting the substrate to the product at a rate that is at least 1.5-times, 2-times, 3-times, 4-times, 5-times, 10-times, 25-times, 50-times, 100-times, 150-times, 200-times, 400-times, 1000-times, 3000-times, 5000-times, 7000-times or more than 7000-times the rate exhibited by the enzyme of SEQ ID NO: 1.

In some embodiments, such ketoreductase polypeptides are also capable of converting the substrate to the product with a percent stereometric excess of at least about 80%. In some embodiments, such ketoreductase polypeptides are also capable of converting the substrate to the product with a percent stereometric excess of at least about 90%. In some embodiments, such ketoreductase polypeptides are also capable of converting the substrate to the product with a percent stereometric excess of at least about 99%.

In some embodiments, the ketoreductase polypeptide is highly stereoselective, wherein the polypeptide can reduce the substrate to the product in greater than about 99%, 99.1%, 99.2%, 99.3%, 99.4%, 99.5%, 99.6%, 99.7%, 99.8%, or 99.9% stereometric excess.

In some embodiments, an improved ketoreductase polypeptide of the disclosure is a polypeptide that comprises an amino acid sequence having at least 80% sequence identity to SEQ ID NO: 2 which has the amino acid sequence as set forth below:

(SEQ ID NO: 2)
MKKIDNAVLPAGSLVLVTGANGFVGSHVVEQLLEHGYKVRGTARSASKL
ANLQKRWDAKYPGRFETAVVEDMLKDGAYDEVIKGAAGVAHIASPVSFS
PKYDEVVPPAIGGTLNALRAAAATPSVKRFVLTSSMMAAIVPKPNVPGI
YLDEKSWNLESIDKAVTLPESHKEKGLWVYAASKTMAEMLAWHFMDENK
PHFTLNTVLPAYTIGTIFDPETQSGSTSGIVMKLFNGEVSPMLALFGPQ
HYVSAFDIGLLHLGCLVLPQIERRRVYGTAAPFDWNMVLAVFRKLWPSK
TFPADFPDQGQDLSVFDTRPSLEILKSLGREGWRSFEDSIKDLVGSEFD
GQTGHHHHHH

In some embodiments, an improve ketoreductase polypeptide of the disclosure is based on the sequence formulas of SEQ ID NO: 2 and can comprise an amino acid sequence that is at least 85%, 86%, 87%, 88%, 89%, 90%, 91%, 92%, 93%, 94%, 95%, 96%, 97%, 98%, or 99% identical to the reference sequence of SEQ ID NO: 2.

These differences between these variants and SEQ ID NO:2 can be amino acid insertions, deletions, substitutions, or any combinations of such changes. In some embodiments, the amino acid sequence differences can comprise non-conservative, conservative, as well as a combination of non-conservative and conservative amino acid substitutions.

In some embodiments, the ketoreductase polypeptide is a polypeptide selected from SEQ ID NOs: 2, 8, 9, 10, 11, 12, 13, 14, 15, and 16. In specific embodiments, the ketoreductase polypeptide is a polypeptide selected from SEQ ID NOs: 2, 14, 15, and 16.

In particular embodiments, an improved ketoreductase polypeptide of the disclosure wherein the amino acid sequence consists of SEQ ID NO: 2. In specific embodiments, the improved ketoreductase polypeptide of the disclosure consists of SEQ ID NO: 2.

In one aspect, an improved ketoreductase polypeptide of the disclosure is based on an amino acid sequence having at least 80% sequence identity to SEQ ID NO: 2, wherein at least at least one of the following conditions are satisfied:
(a) amino acid (aa) residue 2 of SEQ ID NO:2 is other than alanine, or
(b) aa residue 11 of SEQ ID NO:2 is other than glutamic acid.
   In certain embodiments, both conditions (a) and (b) are satisfied in the improved ketoreductase polypeptide of the disclosure. In particular embodiments, aa residue 2 of SEQ ID NO:2 is lysine in the improved ketoreductase polypeptide of the disclosure. In specific embodiments, aa residue 11 of SEQ ID NO:2 is alanine in the improved ketoreductase polypeptide of the disclosure.

In certain embodiments of this aspect, the improved ketoreductase polypeptide of the disclosure is based on an amino acid sequence having at least 85%, 90%, 95%, or 98% sequence identity to SEQ ID NO: 2.

The Applicant has discovered that polypeptides having an aa residue other than alanine residue at the position corresponding to position 2 of SEQ ID NO:2 are better expressed from their parent polynucleotide. Similarly, the Applicant has found that polypeptides having an aa residue other than glutamic acid residue at the position corresponding to position 11 of SEQ ID NO:2 are also better expressed from their parent polynucleotide.

Additional embodiments provide host cells comprising the polynucleotides and/or expression vectors described herein. The host cells may be E. coli, or they may be a different organism, such as L. brevis. The host cells can be used for the expression and isolation of the ketoreductase enzymes described herein, or, alternatively, they can be used directly for the conversion of the substrate to the stereoisomeric product.

Whether carrying out the method with whole cells, cell extracts or purified ketoreductase enzymes, a single ketoreductase enzyme may be used or, alternatively, mixtures of two or more ketoreductase enzymes may be used.

Embodiments relate to ketoreductase enzymes capable of selectively preparing chiral alcohols in the synthesis of indanediols, particularly fluoro-substituted indanediols. In embodiments, the ketoreductase enzymes are capable of the following conversion:

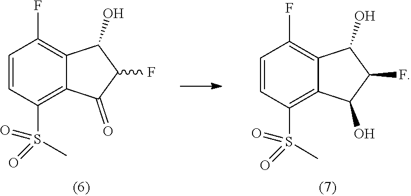 [SMILES: CS(=O)(=O)c1ccc(F)c2c1C(=O)C(F)[C@H]2O ; CS(=O)(=O)c1ccc(F)c2c1[C@H](O)[C@H](F)[C@H]2O]

In particular embodiments, the ketoreductase enzymes in conjunction with electrophilic fluorinating agents are capable of the following conversion:

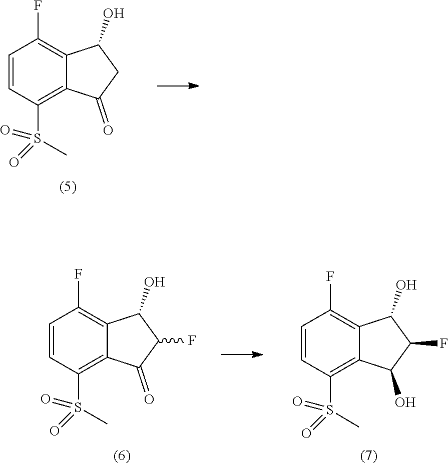 [SMILES: CS(=O)(=O)c1ccc(F)c2c1C(=O)C[C@H]2O ; CS(=O)(=O)c1ccc(F)c2c1C(=O)C(F)[C@H]2O ; CS(=O)(=O)c1ccc(F)c2c1[C@H](O)[C@H](F)[C@H]2O]

Enzyme properties for which improvements are desirable include, but are not limited to, enzymatic activity, thermal stability, pH activity profile, cofactor requirements, refractoriness to inhibitors (e.g., product inhibition), stereospecificity, stereoselectivity, and solvent stability. The improvements can relate to a single enzyme property, such as enzymatic activity, or a combination of different enzyme properties, such as enzymatic activity and stereoselectivity.

Table 1 below provides a list of the SEQ ID NOs disclosed herein with associated activities. The sequences below are based on the ketoreductase sequence of SEQ ID NO: 1, unless otherwise specified. In Table 1 below, each row lists a SEQ ID NO. The column listing the number of mutations (i.e., residue changes) refers to the number of amino acid substitutions as compared to the ketoreductase sequence of SEQ ID NO: 1. In the Activity column of Table 1, + indicates 30-50% (v/v) untreated lysate, ++ indicates 15-30% (v/v) untreated lysate, +++ indicates 15-30% iPrOH-treated lysate, ++++ indicates 10-15% iPrOH-treated lysate, +++++ indicates 5-10% iPrOH-treated lysate. In the Selectivity column of the Table, + indicates a total diastereomeric ratio<10:1, ++ indicates a total dr between 10:1 and 25:1, +++ indicates a total dr between 25:1 and 50:1, ++++ indicates a total dr between 50:1 and 99:1, +++++ indicates a total dr>99:1. In the Solvent Tolerance column of the Table, + indicates 0-12.5% (v/v) total organic solvent, ++ indicates 12.5-18% (v/v) total organic solvent, +++ indicates 18-25% (v/v) total organic solvent, ++++ indicates 25-40% (v/v) total organic solvent, +++++ indicates 40-50% (v/v) total organic solvent present. In the Volumetric Productivity column of the Table, + indicates 0-20 g/L of purified substrate, ++ indicates 20-40 g/L of crude substrate, +++ indicates 40-50 g/L of crude substrate, ++++ indicates 50-60 g/L of crude subtrate, +++++ indicates 60-80 g/L of crude substrate. In the iPrOH Utilization column of the Table, + indicates 7.5-10% (v/v), ++ indicates 11-13% (v/v), +++ indicates 13.5% (v/v) iPrOH with 1.5% (v/v) acetone, ++++ indicates 15% (v/v) iPrOH, +++++ indicates 16-18% (v/v) iPrOH.

The level of improvement for the properties noted Table 1 were determined by setting up reactions with increasingly challenging conditions, including increasing organic solvent (both of composition and amount) and increasing substrate concentration. Each enzyme variant's performance was measured by observing the amount of product formed on an Agilent UPLC instrument, typically after an overnight reaction as described in Example 2. When screening focused on improving diastereoselectivity, the amounts of each of the products formed was measured by an Agilent SFC instrument, also described in Example 2.

TABLE 1
Activity of Ketoreductase Enzymes

| | Residue | Number of | | | | | |
|---|---|---|---|---|---|---|---|
| | Changes | Changes | | | | | |
| SEQ | Relative | Relative | | | | | |
| ID | to SEQ | to SEQ | | | Solvent | Volumetric | iPrOH |
| NO: | ID NO: 1 | ID NO: 1 | Activity | Selectivity | Tolerance | Productivity | Utilization |
| 1 | — | — | − | − | − | − | − |
| 4 | L205N, | 2 | + | + | + | − | + |
| | M242V | | | | | | |
| 5 | V94P, | 4 | + | ++ | ++ | + | + |
| | V135M, | | | | | | |
| | L205N, | | | | | | |
| | M242V | | | | | | |
| 6 | A2K, | 5 | ++ | ++ | ++ | + | − |
| | V94P, | | | | | | |
| | V135M, | | | | | | |
| | L205N, | | | | | | |
| | M242V | | | | | | |
| 7 | A2K, | 7 | ++ | +++ | +++ | + | − |
| | E11A, | | | | | | |
| | V94P, | | | | | | |
| | V135M, | | | | | | |
| | L205N, | | | | | | |
| | M242F, | | | | | | |
| | P243R | | | | | | |
| 8 | A2K, | 13 | ++ | ++++ | +++ | ++ | +++ |
| | E11A, | | | | | | |
| | V94P, | | | | | | |
| | V135M, | | | | | | |
| | S136A, | | | | | | |
| | Q171Y, | | | | | | |
| | S173G, | | | | | | |
| | A187L, | | | | | | |
| | L205N, | | | | | | |
| | S229K, | | | | | | |
| | M242F, | | | | | | |
| | P243R, | | | | | | |
| | I330F | | | | | | |
| 9 | A2K, | 17 | ++ | ++++ | +++ | ++ | +++ |
| | E11A, | | | | | | |
| | V94P, | | | | | | |
| | V135M, | | | | | | |
| | S136A, | | | | | | |
| | I139V, | | | | | | |
| | Q171Y, | | | | | | |
| | S173G, | | | | | | |
| | E183T, | | | | | | |
| | A187L, | | | | | | |
| | L205A, | | | | | | |
| | N207A, | | | | | | |
| | W226F, | | | | | | |
| | S229K, | | | | | | |
| | M242F, | | | | | | |
| | P243R, | | | | | | |
| | I330F | | | | | | |
| 10 | A2K, | 20 | ++ | +++ | ++++ | ++ | +++ |
| | E11A, | | | | | | |
| | V94P, | | | | | | |
| | V135M, | | | | | | |
| | S136A, | | | | | | |
| | I139V, | | | | | | |
| | Q171Y, | | | | | | |
| | S173G, | | | | | | |
| | E183T, | | | | | | |
| | A187L, | | | | | | |
| | L205A, | | | | | | |
| | N207A, | | | | | | |
| | W226F, | | | | | | |
| | S229K, | | | | | | |
| | A238M, | | | | | | |
| | M242F, | | | | | | |
| | P243A, | | | | | | |
| | T282G, | | | | | | |
| | Y291W, | | | | | | |
| | I330F | | | | | | |
| 11 | A2K, | 22 | +++ | ++ | ++++ | +++ | +++++ |
| | E11A, | | | | | | |
| | V94P, | | | | | | |
| | V135M, | | | | | | |
| | S136A, | | | | | | |
| | I139V, | | | | | | |
| | K163V, | | | | | | |
| | Q171E, | | | | | | |
| | S173G, | | | | | | |
| | E183T, | | | | | | |
| | A187L, | | | | | | |
| | L205A, | | | | | | |
| | N207A, | | | | | | |
| | W226F, | | | | | | |
| | S229K, | | | | | | |
| | A238M, | | | | | | |
| | M242F, | | | | | | |
| | R243G, | | | | | | |
| | T282G, | | | | | | |
| | Y291W, | | | | | | |
| | I330F, | | | | | | |
| | E332D | | | | | | |
| 12 | A2K, | 26 | +++ | +++ | ++++ | +++ | +++++ |
| | E11A, | | | | | | |
| | V94P, | | | | | | |
| | S96L, | | | | | | |
| | N99P, | | | | | | |
| | V135M, | | | | | | |
| | S136A, | | | | | | |
| | I139V, | | | | | | |
| | K163V, | | | | | | |
| | P170K, | | | | | | |
| | Q171E, | | | | | | |
| | S173G, | | | | | | |
| | E183T, | | | | | | |
| | L186M, | | | | | | |
| | A187L, | | | | | | |
| | A203T, | | | | | | |
| | N207A, | | | | | | |
| | W226F, | | | | | | |
| | S229K, | | | | | | |
| | A238M, | | | | | | |
| | M242F, | | | | | | |
| | R243G, | | | | | | |
| | T282G, | | | | | | |
| | Y291W, | | | | | | |
| | E332D, | | | | | | |
| | I330F | | | | | | |
| 13 | A2K, | 31 | +++ | ++++ | +++++ | +++ | +++++ |
| | E11A, | | | | | | |
| | V94P, | | | | | | |
| | S96L, | | | | | | |
| | N99P, | | | | | | |
| | V135M, | | | | | | |
| | S136A, | | | | | | |
| | I139V, | | | | | | |
| | K163V, | | | | | | |
| | P170K, | | | | | | |
| | Q171E, | | | | | | |
| | S173G, | | | | | | |
| | E183T, | | | | | | |
| | L186M, | | | | | | |
| | A187L, | | | | | | |
| | A203T, | | | | | | |
| | N207A, | | | | | | |
| | W226F, | | | | | | |
| | M227V, | | | | | | |
| | S229K, | | | | | | |
| | A238M, | | | | | | |
| | M242F, | | | | | | |
| | R243G, | | | | | | |
| | Y246H, | | | | | | |
| | T282G, | | | | | | |
| | Y291W, | | | | | | |
| | A313T, | | | | | | |
| | I330F, | | | | | | |
| | E332D, | | | | | | |
| | T342F, | | | | | | |
| | A343D | | | | | | |
| 14 | A2K, | 37 | ++++ | +++++ | +++++ | +++ | +++++ |
| | E11A, | | | | | | |
| | V94P, | | | | | | |
| | S96L, | | | | | | |
| | S98Q, | | | | | | |
| | N99P, | | | | | | |
| | T134M, | | | | | | |
| | V135M, | | | | | | |
| | S136A, | | | | | | |
| | L138Y, | | | | | | |
| | I139V, | | | | | | |
| | E145Q, | | | | | | |
| | K163V, | | | | | | |
| | P170K, | | | | | | |
| | Q171E, | | | | | | |
| | S173G, | | | | | | |
| | E183M, | | | | | | |
| | L186M, | | | | | | |
| | A187L, | | | | | | |
| | A203T, | | | | | | |
| | N207A, | | | | | | |
| | W226I, | | | | | | |
| | M227V, | | | | | | |
| | S229K, | | | | | | |
| | A238M, | | | | | | |
| | M242V, | | | | | | |
| | R243G, | | | | | | |
| | Y246H, | | | | | | |
| | T277P, | | | | | | |
| | T282M, | | | | | | |
| | Y291W, | | | | | | |
| | K309V, | | | | | | |
| | A313R, | | | | | | |
| | I330F, | | | | | | |
| | E332D, | | | | | | |
| | T342F, | | | | | | |
| | A343D | | | | | | |
| 15 | A2K, | 37 | +++++ | +++++ | ++++ | ++++ | +++++ |
| | E11A, | | | | | | |
| | V94P, | | | | | | |
| | N99P, | | | | | | |
| | T134M, | | | | | | |
| | V135M, | | | | | | |
| | S136A, | | | | | | |
| | L138Y, | | | | | | |
| | I139V, | | | | | | |
| | E145Q, | | | | | | |
| | K163P, | | | | | | |
| | D196H, | | | | | | |
| | P170K, | | | | | | |
| | Q171E, | | | | | | |
| | S173G, | | | | | | |
| | E183M, | | | | | | |
| | L186M, | | | | | | |
| | A187L, | | | | | | |
| | A203T, | | | | | | |
| | N207A, | | | | | | |
| | W226I, | | | | | | |
| | M227V, | | | | | | |
| | S229K, | | | | | | |
| | A238M, | | | | | | |
| | M242F, | | | | | | |
| | R243G, | | | | | | |
| | Y246H, | | | | | | |
| | T277P, | | | | | | |
| | T282M, | | | | | | |
| | Y291W, | | | | | | |
| | K309V, | | | | | | |
| | A313R, | | | | | | |
| | P325E, | | | | | | |
| | I330F, | | | | | | |
| | E332D, | | | | | | |
| | T342F, | | | | | | |
| | A343D | | | | | | |
| 16 | A2K, | 38 | +++++ | +++++ | +++++ | +++++ | +++++ |
| | E11A, | | | | | | |
| | Q75D, | | | | | | |
| | V94P, | | | | | | |
| | N99P, | | | | | | |
| | T106P, | | | | | | |
| | T134M, | | | | | | |
| | V135M, | | | | | | |
| | S136A, | | | | | | |
| | L138I, | | | | | | |
| | I139V, | | | | | | |
| | E145Q, | | | | | | |
| | K163V, | | | | | | |
| | P170K, | | | | | | |
| | Q171E, | | | | | | |
| | S173G, | | | | | | |
| | E183M, | | | | | | |
| | L186M, | | | | | | |
| | A187L, | | | | | | |
| | A203T, | | | | | | |
| | N207A, | | | | | | |
| | W226I, | | | | | | |
| | M227V, | | | | | | |
| | S229K, | | | | | | |
| | A238M, | | | | | | |
| | M242F, | | | | | | |
| | R243G, | | | | | | |
| | Y246H, | | | | | | |
| | V251F, | | | | | | |
| | T277P, | | | | | | |
| | T282M, | | | | | | |
| | Y291W, | | | | | | |
| | K309V, | | | | | | |
| | A313R, | | | | | | |
| | I330F, | | | | | | |
| | E332D, | | | | | | |
| | T342F, | | | | | | |
| | A343D | | | | | | |
| 2 | A2K, | 43 | +++++ | ++++ | +++++ | +++++ | +++++ |
| | E11A, | | | | | | |
| | Q75D, | | | | | | |
| | V94P, | | | | | | |
| | N99P, | | | | | | |
| | T106P, | | | | | | |
| | T134M, | | | | | | |
| | V135M, | | | | | | |
| | S136A, | | | | | | |
| | L138I, | | | | | | |
| | I139V, | | | | | | |
| | E145P, | | | | | | |
| | K163V, | | | | | | |
| | D196H, | | | | | | |
| | P170K, | | | | | | |
| | Q171E, | | | | | | |
| | S173G, | | | | | | |
| | E183M, | | | | | | |
| | L186M, | | | | | | |
| | A187L, | | | | | | |
| | K190H, | | | | | | |
| | A203T, | | | | | | |
| | N207A, | | | | | | |
| | W226I, | | | | | | |
| | M227V, | | | | | | |
| | S229K, | | | | | | |
| | A238M, | | | | | | |
| | M242F, | | | | | | |
| | R243G, | | | | | | |
| | Y246H, | | | | | | |
| | V251F, | | | | | | |
| | G276A, | | | | | | |
| | T277P, | | | | | | |
| | T282M, | | | | | | |
| | T286V, | | | | | | |
| | Y291W, | | | | | | |
| | K309V, | | | | | | |
| | A313R, | | | | | | |
| | P325E, | | | | | | |
| | I330F, | | | | | | |
| | E332D, | | | | | | |
| | T342F, | | | | | | |
| | A343D | | | | | | |

### Polynucleotides Encoding Ketoreductases

In another aspect, the present disclosure provides polynucleotides encoding the ketoreductase polypeptides disclosed herein. The polynucleotides may be operatively linked to one or more heterologous regulatory sequences that control gene expression to create a recombinant polynucleotide capable of expressing the polypeptide. Expression constructs containing a heterologous polynucleotide encoding the ketoreductase can be introduced into appropriate host cells to express the corresponding ketoreductase polypeptide.

Because of the knowledge of the codons corresponding to the various amino acids, availability of a protein sequence provides a description of all the polynucleotides capable of encoding the subject. The degeneracy of the genetic code, where the same amino acids are encoded by alternative or synonymous codons allows an extremely large number of nucleic acids to be made, all of which encode the improved ketoreductase enzymes disclosed herein. Thus, having identified a particular amino acid sequence, those skilled in the art could make any number of different nucleic acids by simply modifying the sequence of one or more codons in a way that does not change the amino acid sequence of the protein. In this regard, the present disclosure specifically contemplates each and every possible variation of polynucleotides that could be made by selecting combinations based on the possible codon choices, and all such variations are to be considered specifically disclosed for any polypeptide disclosed herein.

In various embodiments, the codons are preferably selected to fit the host cell in which the protein is being produced. For example, preferred codons used in bacteria are used to express the gene in bacteria; preferred codons used in yeast are used for expression in yeast; and preferred codons used in mammals are used for expression in mammalian cells. By way of example, the polynucleotide of SEQ ID NO: 3 (vide infra) which encodes SEQ ID NO:2 has been codon optimized for expression in E. coli.

In another aspect, the Applicant has discovered that certain changes to the triplet codons in the polynucleotides encoding the ketoreductase polypeptides of the disclosure confer an increased expression of the polypeptides. These changes typically occur in the codons encoding amino acid residues toward the N-terminus of the polypeptide. For instance, by reference to the polynucleotide of SEQ ID NO: 17 (vide infra) which encodes SEQ ID NO:1, the Applicant notes that changing the codons encoding the alanine residue at position 2 (GCT), the lysine residue at position 3 (AAA), the isoleucine residue at position 4 (ATC) and the glutamic acid residue at position 11 (GAA) of the polypeptide an increased expression of the polypeptide is conferred. In some embodiments, the change in the triplet codon at such positions does not result in an amino acid residue change in the encoded polypeptide. In other embodiments, the change in the triplet codon at such positions does result in an amino acid residue change in the encoded polypeptide. Accordingly, in one embodiment, the present disclosure provides a polynucleotide encoding a polypeptide having at least 80% or 90% identity to SEQ ID NO:2, wherein at least one of the following conditions are satisfied:
(a) the triplet codon encoding the amino acid residue at position 2 of the polypeptide is other than GCT;
(b) the triplet codon encoding the amino acid residue at position 3 of the polypeptide is other than AAA;
(c) the triplet codon encoding the amino acid residue at position 4 of the polypeptide is other than ATC; or
(d) the triplet codon encoding the amino acid residue at position 11 of the polypeptide is other than GAA.

In some embodiments at least two of the conditions (a)-(d) are satisfied. In certain embodiments at least three conditions of the conditions (a)-(d) are satisfied. In specific embodiments all four of the conditions of (a)-(d) are satisfied.

In certain embodiments, all codons need not be replaced to optimize the codon usage of the ketoreductases since the natural sequence will comprise preferred codons and because use of preferred codons may not be required for all amino acid residues. Consequently, codon optimized polynucleotides encoding the ketoreductase enzymes may contain preferred codons at about 40%, 50%, 60%, 70%, 80%, or greater than 90% of codon positions of the full length coding region.

In various embodiments, an isolated polynucleotide encoding an improved ketoreductase polypeptide may be manipulated in a variety of ways to provide for expression of the polypeptide. Manipulation of the isolated polynucleotide prior to its insertion into a vector may be desirable or necessary depending on the expression vector. The techniques for modifying polynucleotides and nucleic acid sequences utilizing recombinant DNA methods are well known in the art. Guidance is provided in Sambrook et al., 2001, Molecular Cloning: A Laboratory Manual, 3rd Ed., Cold Spring Harbor Laboratory Press; and Current Protocols in Molecular Biology, Ausubel. F. ed., Greene Pub. Associates, 1998, updates to 2006.

In some embodiments, an isolated polynucleotide encoding any of the ketoreductase polypeptides herein is manipulated in a variety of ways to facilitate expression of the ketoreductase polypeptide. In some embodiments, the polynucleotides encoding the ketoreductase polypeptides comprise expression vectors where one or more control sequences is present to regulate the expression of the ketoreductase polynucleotides and/or polypeptides. Manipulation of the isolated polynucleotide prior to its insertion into a vector may be desirable or necessary depending on the expression vector utilized. Techniques for modifying polynucleotides and nucleic acid sequences utilizing recombinant DNA methods are well known in the art. In some embodiments, the control sequences include among others, promoters, leader sequences, polyadenylation sequences, propeptide sequences, signal peptide sequences, and transcription terminators. In some embodiments, suitable promoters are selected based on the host cells selection. For bacterial host cells, suitable promoters for directing transcription of the nucleic acid constructs of the present disclosure, include, but are not limited to, promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (See e.g., Villa-Kamaroff et al., Proc. Natl Acad. Sci. USA 75: 3727-3731 [1978]), as well as the tac promoter (See e.g., DeBoer et al., Proc. Natl Acad. Sci. USA 80: 21-25 [1983]). Exemplary promoters for filamentous fungal host cells, include, but are not limited to, promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (See e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Exemplary yeast cell promoters can be from the genes can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are known in the art (See e.g., Romanos et al., Yeast 8:423-488 [1992]).

In some embodiments, the control sequence is also a suitable transcription terminator sequence (i.e., a sequence recognized by a host cell to terminate transcription). In some embodiments, the terminator sequence is operably linked to the 3′ terminus of the nucleic acid sequence encoding the enzyme polypeptide. Any suitable terminator that is functional in the host cell of choice finds use in the present invention. Exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are known in the art (See e.g., Romanos et al., supra).

In some embodiments, the control sequence is also a suitable leader sequence (i.e., a non-translated region of an mRNA that is important for translation by the host cell). In some embodiments, the leader sequence is operably linked to the 5′ terminus of the nucleic acid sequence encoding the ketoreductase. Any suitable leader sequence that is functional in the host cell of choice find use in the present invention. Exemplary leaders for filamentous fungal host cells are obtained from the genes for Aspergillus oryzae TAKA amylase, and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

In some embodiments, the control sequence is also a polyadenylation sequence (i.e., a sequence operably linked to the 3′ terminus of the nucleic acid sequence and which, when transcribed, is recognized by the host cell as a signal to add polyadenosine residues to transcribed mRNA). Any suitable polyadenylation sequence that is functional in the host cell of choice may be used in the present invention. Exemplary polyadenylation sequences for filamentous fungal host cells include, but are not limited to, the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are known (See e.g., Guo and Sherman, Mol. Cell. Biol., 15:5983-5990 [1995]).

In some embodiments, the control sequence is also a signal peptide (i.e., a coding region that codes for an amino acid sequence linked to the amino terminus of a polypeptide and directs the encoded polypeptide into the cell's secretory pathway). In some embodiments, the 5′ end of the coding sequence of the nucleic acid sequence inherently contains a signal peptide coding region naturally linked in translation reading frame with the segment of the coding region that encodes the secreted polypeptide. Alternatively, in some embodiments, the 5′ end of the coding sequence contains a signal peptide coding region that is foreign to the coding sequence. Any suitable signal peptide coding region that directs the expressed polypeptide into the secretory pathway of a host cell of choice finds use for expression of the engineered polypeptide(s). Effective signal peptide coding regions for bacterial host cells are the signal peptide coding regions include, but are not limited to, those obtained from the genes for Bacillus NC1B 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are known in the art (See e.g., Simonen and Palva, Microbiol. Rev., 57:109-137 [1993]). In some embodiments, effective signal peptide coding regions for filamentous fungal host cells include, but are not limited to, the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase. Useful signal peptides for yeast host cells include, but are not limited to, those from the genes for Saccharomyces cerevisiae alpha-factor and Saccharomyces cerevisiae invertase.

In some embodiments, regulatory sequences are also utilized. These sequences facilitate the regulation of the expression of the polypeptide relative to the growth of the host cell. Examples of regulatory systems are those that cause the expression of the gene to be turned on or off in response to a chemical or physical stimulus, including the presence of a regulatory compound. In prokaryotic host cells, suitable regulatory sequences include, but are not limited to, the lac, tac, and trp operator systems. In yeast host cells, suitable regulatory systems include, but are not limited to, the ADH2 system or GAL1 system. In filamentous fungi, suitable regulatory sequences include, but are not limited to, the TAKA alpha-amylase promoter, Aspergillus niger glucoamylase promoter, and Aspergillus oryzae glucoamylase promoter.

In another aspect, the present invention is directed to a recombinant expression vector comprising a polynucleotide encoding ketoreductase polypeptide, and one or more expression regulating regions such as a promoter and a terminator, a replication origin, etc., depending on the type of hosts into which they are to be introduced. In some embodiments, the various nucleic acid and control sequences described herein are joined together to produce recombinant expression vectors that include one or more convenient restriction sites to allow for insertion or substitution of the nucleic acid sequence encoding the enzyme polypeptide at such sites. Alternatively, in some embodiments, the nucleic acid sequence of the present invention is expressed by inserting the nucleic acid sequence or a nucleic acid construct comprising the sequence into an appropriate vector for expression. In some embodiments involving the creation of the expression vector, the coding sequence is located in the vector so that the coding sequence is operably linked with the appropriate control sequences for expression.

The recombinant expression vector may be any suitable vector (e.g., a plasmid or virus), that can be conveniently subjected to recombinant DNA procedures and bring about the expression of the enzyme polynucleotide sequence. The choice of the vector typically depends on the compatibility of the vector with the host cell into which the vector is to be introduced. The vectors may be linear or closed circular plasmids.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome). The vector may contain any means for assuring self-replication. In some alternative embodiments, the vector is one in which, when introduced into the host cell, it is integrated into the genome and replicated together with the chromosome(s) into which it has been integrated. Furthermore, in some embodiments, a single vector or plasmid, or two or more vectors or plasmids which together contain the total DNA to be introduced into the genome of the host cell, and/or a transposon is utilized.

In some embodiments, the expression vector contains one or more selectable markers, which permit easy selection of transformed cells. A “selectable marker” is a gene, the product of which provides for biocide or viral resistance, resistance to heavy metals, prototrophy to auxotrophs, and the like. Examples of bacterial selectable markers include, but are not limited to, the dal genes from Bacillus subtilis or Bacillus licheniformis, or markers, which confer antibiotic resistance such as ampicillin, kanamycin, chloramphenicol or tetracycline resistance. Suitable markers for yeast host cells include, but are not limited to, ADE2, HIS3, LEU2, LYS2, MET3, TRP1, and URA3. Selectable markers for use in filamentous fungal host cells include, but are not limited to, amdS (acetamidase; e.g., from A. nidulans or A. orzyae), argB (ornithine carbamoyltransferases), bar (phosphinothricin acetyltransferase; e.g., from S. hygroscopicus), hph (hygromycin phosphotransferase), niaD (nitrate reductase), pyrG (orotidine-5′-phosphate decarboxylase; e.g., from A. nidulans or A. orzyae), sC (sulfate adenyltransferase), and trpC (anthranilate synthase), as well as equivalents thereof.

In another aspect, the present invention provides a host cell comprising at least one polynucleotide encoding at least one ketoreductase of the present disclosure, the polynucleotide(s) being operatively linked to one or more control sequences for expression of the at least one ketoreductase in the host cell. Host cells suitable for use in expressing the polypeptides encoded by the expression vectors of the present invention are well known in the art and include but are not limited to, bacterial cells, such as E. coli, Vibrio fluvialis, Streptomyces and Salmonella typhimurium cells; fungal cells, such as yeast cells (e.g., Saccharomyces cerevisiae or Pichia pastoris (ATCC Accession No. 201178));. Exemplary host cells also include various Escherichia coli strains (e.g., W3110 (ΔfhuA) and BL21). Examples of bacterial selectable markers include, but are not limited to, the dal genes from Bacillus subtilis or Bacillus licheniformis, or markers, which confer antibiotic resistance such as ampicillin, kanamycin, chloramphenicol, and or tetracycline resistance.

In some embodiments, the expression vectors of the present invention contain an element(s) that permits integration of the vector into the host cell's genome or autonomous replication of the vector in the cell independent of the genome. In some embodiments involving integration into the host cell genome, the vectors rely on the nucleic acid sequence encoding the polypeptide or any other element of the vector for integration of the vector into the genome by homologous or nonhomologous recombination.

In some alternative embodiments, the expression vectors contain additional nucleic acid sequences for directing integration by homologous recombination into the genome of the host cell. The additional nucleic acid sequences enable the vector to be integrated into the host cell genome at a precise location(s) in the chromosome(s). To increase the likelihood of integration at a precise location, the integrational elements preferably contain a sufficient number of nucleotides, such as 100 to 10,000 base pairs, preferably 400 to 10,000 base pairs, and most preferably 800 to 10,000 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences. On the other hand, the vector may be integrated into the genome of the host cell by non-homologous recombination.

For autonomous replication, the vector may further comprise an origin of replication enabling the vector to replicate autonomously in the host cell in question. Examples of bacterial origins of replication are P15A on or the origins of replication of plasmids pBR322, pUC19, pACYC177 (which contains the P15A on), or pACYC184 (which contains the P15A on) permitting replication in E. coli, and pUB110, pE194, or pTA1060 permitting replication in Bacillus. Examples of origins of replication for use in a yeast host cell are the 2 micron origin of replication, ARS1, ARS4, the combination of ARS1 and CEN3, and the combination of ARS4 and CEN6. The origin of replication may be one having a mutation which makes its functioning temperature-sensitive in the host cell (See e.g., Ehrlich, Proc. Natl. Acad. Sci. USA 75:1433 [1978]).

In some embodiments, more than one copy of a nucleic acid sequence of the present invention is inserted into the host cell to increase production of the gene product. An increase in the copy number of the nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent.

Many of the expression vectors for use in the present invention are commercially available. Suitable commercial expression vectors include, but are not limited to, Novagen's® pET E. coli T7 expression vectors (Millipore Sigma) and the p3×FLAGTM™ expression vectors (Sigma-Aldrich Chemicals). Other suitable expression vectors include, but are not limited to, pBluescriptII SK(−) and pBK-CMV (Stratagene), and plasmids derived from pBR322 (Gibco BRL), pUC (Gibco BRL), pREP4, pCEP4 (Invitrogen) or pPoly (See e.g., Lathe et al., Gene 57:193-201 [1987]).

Thus, in some embodiments, a vector comprising a sequence encoding at least one variant ketoreductase is transformed into a host cell in order to allow propagation of the vector and expression of the variant ketoreductase(s). In some embodiments, the transformed host cell described above is cultured in a suitable nutrient medium under conditions permitting the expression of the variant ketoreductase(s). Any suitable medium useful for culturing the host cells finds use in the present invention, including, but not limited to minimal or complex media containing appropriate supplements. In some embodiments, host cells are grown in HTP media. Suitable media are available from various commercial suppliers or may be prepared according to published recipes (e.g., in catalogues of the American Type Culture Collection).

### Host Cells for Expression of Ketoreductases

In another aspect, the present disclosure provides a host cell comprising a polynucleotide encoding an improved ketoreductase polypeptide of the present disclosure, the polynucleotide being operatively linked to one or more control sequences for expression of the ketoreductase enzyme in the host cell. Host cells for use in expressing the ketoreductase polypeptides encoded by the expression vectors of the present invention are well known in the art and include but are not limited to, bacterial cells, such as E. coli, B. subtilis, B. licheniformis, B. megaterium, B. stearothermophilus, B. amyloliquefaciens, Lactobacillus kejir, Lactobacillus brevis, Lactobacillus minor, Streptomyces and Salmonella typhimurium cells; fungal cells, such as yeast cells (e.g., Saccharomyces cerevisiae or Pichia pastoris (ATCC Accession No. 201178)). Appropriate culture mediums and growth conditions for the above-described host cells are well known in the art.

Polynucleotides for expression of the ketoreductases may be introduced into cells by various methods known in the art. Techniques include among others, electroporation, biolistic particle bombardment, liposome mediated transfection, calcium chloride transfection, and protoplast fusion. Various methods for introducing polynucleotides into cells will be apparent to the skilled artisan.

In some embodiments of the present invention, the filamentous fungal host cells are of any suitable genus and species, including, but not limited to Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothis, Fusarium, Gibberella, Gliocladium, Humicola, Hypocrea, Myceliophthora, Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Trametes, Tolypocladium, Trichoderma, Verticillium, and/or Volvariella, and/or teleomorphs, or anamorphs, and synonyms, basionyms, or taxonomic equivalents thereof.

In some embodiments of the present invention, the host cell is a yeast cell, including but not limited to cells of Candida, Hansenula, Saccharomyces, Schizosaccharomyces, Pichia, Kluyveromyces, or Yarrowia species. In some embodiments of the present invention, the yeast cell is Hansenula polymorpha, Saccharomyces cerevisiae, Saccharomyces carlsbergensis, Saccharomyces diastaticus, Saccharomyces norbensis, Saccharomyces kluyveri, Schizosaccharomyces pombe, Pichia pastoris, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, or Yarrowia lipolytica.

In some other embodiments, the host cell is a prokaryotic cell. Suitable prokaryotic cells include, but are not limited to, Gram-positive, Gram-negative and Gram-variable bacterial cells. Any suitable bacterial organism finds use in the present invention, including but not limited to Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia and Zymomonas. In some embodiments, the host cell is a species of Agrobacterium, Acinetobacter, Azobacter, Bacillus, Bifidobacterium, Buchnera, Geobacillus, Campylobacter, Clostridium, Corynebacterium, Escherichia, Enterococcus, Erwinia, Flavobacterium, Lactobacillus, Lactococcus, Pantoea, Pseudomonas, Staphylococcus, Salmonella, Streptococcus, Streptomyces, or Zymomonas. In some embodiments, the bacterial host strain is non-pathogenic to humans. In some embodiments the bacterial host strain is an industrial strain. Numerous bacterial industrial strains are known and suitable in the present invention. In some embodiments of the present invention, the bacterial host cell is an Agrobacterium species (e.g., A. radiobacter, A. rhizogenes, and A. rubi). In some embodiments of the present invention, the bacterial host cell is an Arthrobacter species (e.g., A. aurescens, A. citreus, A. globiformis, A. hydrocarboglutamicus, A. mysorens, A. nicotianae, A. paraffineus, A. protophonniae, A. roseoparqffinus, A. sulfureus, and A. ureafaciens). In some embodiments of the present invention, the bacterial host cell is a Bacillus species (e.g., B. thuringensis, B. anthracis, B. megaterium, B. subtilis, B. lentus, B. circulans, B. pumilus, B. lautus, B. coagulans, B. brevis, B. firmus, B. alkaophius, B. licheniformis, B. clausii, B. stearothermophilus, B. halodurans, and B. amyloliquefaciens). In some embodiments, the host cell is an industrial Bacillus strain including but not limited to B. subtilis, B. pumilus, B. licheniformis, B. megaterium, B. clausii, B. stearothermophilus, or B. amyloliquefaciens. In some embodiments, the Bacillus host cells are B. subtilis, B. licheniformis, B. megaterium, B. stearothermophilus, and/or B. amyloliquefaciens. In some embodiments, the bacterial host cell is a Clostridium species (e.g., C. acetobutylicum, C. tetani E88, C. lituseburense, C. saccharobutylicum, C. perfringens, and C. beijerinckii). In some embodiments, the bacterial host cell is a Corynebacterium species (e.g., C. glutamicum and C. acetoacidophilum). In some embodiments the bacterial host cell is an Escherichia species (e.g., E. coli). In some embodiments, the host cell is Escherichia coli W3110. In some embodiments the host is Escherichia coli BL21 or BL21(DE3). In some embodiments, the bacterial host cell is an Erwinia species (e.g., E. uredovora, E. carotovora, E. ananas, E. herbicola, E. punctata, and E. terreus). In some embodiments, the bacterial host cell is a Pantoea species (e.g., P. citrea, and P. agglomerans). In some embodiments the bacterial host cell is a Pseudomonas species (e.g., P. putida, P. aeruginosa, P. mevalonii, and P. sp. D-01 10). In some embodiments, the bacterial host cell is a Streptococcus species (e.g., S. equisimiles, S. pyogenes, and S. uberis). In some embodiments, the bacterial host cell is a Streptomyces species (e.g., S. ambofaciens, S. achromogenes, S. avermitilis, S. coelicolor, S. aureofaciens, S. aureus, S. fungicidicus, S. griseus, and S. lividans). In some embodiments, the bacterial host cell is a Zymomonas species (e.g., Z. mobilis, and Z. lipolytica).

Many prokaryotic and eukaryotic strains that find use in the present invention are readily available to the public from a number of culture collections such as American Type Culture Collection (ATCC), Deutsche Sammlung von Mikroorganismen und Zellkulturen GmbH (DSM), Centraalbureau Voor Schimmelcultures (CBS), and Agricultural Research Service Patent Culture Collection, Northern Regional Research Center (NRRL).

In some embodiments, host cells are genetically modified to have characteristics that improve protein secretion, protein stability and/or other properties desirable for expression and/or secretion of a protein. Genetic modification can be achieved by genetic engineering techniques and/or classical microbiological techniques (e.g., chemical or UV mutagenesis and subsequent selection). Indeed, in some embodiments, combinations of recombinant modification and classical selection techniques are used to produce the host cells. Using recombinant technology, nucleic acid molecules can be introduced, deleted, inhibited or modified, in a manner that results in increased yields of ketoreductase variant(s) within the host cell and/or in the culture medium. In one genetic engineering approach, homologous recombination is used to induce targeted gene modifications by specifically targeting a gene in vivo to suppress expression of the encoded protein. In alternative approaches, siRNA, antisense and/or ribozyme technology find use in inhibiting gene expression. A variety of methods are known in the art for reducing expression of protein in cells, including, but not limited to deletion of all or part of the gene encoding the protein and site-specific mutagenesis to disrupt expression or activity of the gene product. (See e.g., Chaveroche et al., Nucl. Acids Res., 28:22 e97 [2000]; Cho et al., Molec. Plant Microbe Interact., 19:7-15 [2006]; Maruyama and Kitamoto, Biotechnol. Lett., 30:1811-1817 [2008]; Takahashi et al., Mol. Gen. Genom., 272: 344-352 [2004]; and You et al., Arch. Microbiol., 191:615-622 [2009], all of which are incorporated by reference herein). Random mutagenesis, followed by screening for desired mutations also finds use (See e.g., Combier et al., FEMS Microbiol. Lett., 220:141-8 [2003]; and Firon et al., Eukary. Cell 2:247-55 [2003], both of which are incorporated by reference).

Introduction of a vector or DNA construct into a host cell can be accomplished using any suitable method known in the art, including but not limited to calcium phosphate transfection, DEAE-dextran mediated transfection, PEG-mediated transformation, electroporation, or other common techniques known in the art.

In some embodiments, the engineered host cells (i.e., “recombinant host cells”) of the present invention are cultured in conventional nutrient media modified as appropriate for activating promoters, selecting transformants, or amplifying the ketoreductase polynucleotide. Culture conditions, such as temperature, pH and the like, are those previously used with the host cell selected for expression, and are well-known to those skilled in the art. As noted, many standard references and texts are available for the culture and production of many cells, including cells of bacterial, plant, animal (especially mammalian) and archebacterial origin.

In some embodiments, cells expressing the ketoreductase of the invention are grown under batch or continuous fermentations conditions. Classical “batch fermentation” is a closed system, wherein the compositions of the medium are set at the beginning of the fermentation and is not subject to artificial alternations during the fermentation. A variation of the batch system is a “fed-batch fermentation” that also finds use in the present invention. In this variation, the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art. “Continuous fermentation” is an open system where a defined fermentation medium is added continuously to a bioreactor and an equal amount of conditioned medium is removed simultaneously for processing. Continuous fermentation generally maintains the cultures at a constant high density where cells are primarily in log phase growth. Continuous fermentation systems strive to maintain steady state growth conditions. Methods for modulating nutrients and growth factors for continuous fermentation processes as well as techniques for maximizing the rate of product formation are well known in the art of industrial microbiology.

More than one copy of a nucleic acid sequence of the present invention may be inserted into the host cell to increase production of the gene product. An increase in the copy number of the nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent.

In some embodiments of the present invention, cell-free transcription and translation systems find use in producing the ketoreductase(s). Several systems are commercially available, and the methods are well-known to those skilled in the art.

### Methods of Evolving Ketoreductases

In some embodiments, to make the ketoreductases of the present disclosure, the ketoreductase enzyme that catalyzes the reduction reaction is obtained (or derived) from E. coli. In some embodiments, the parent polynucleotide sequence is codon optimized to enhance expression of the ketoreductase in a specified host cell. The parental polynucleotide sequence, designated as SEQ ID NO: 3, which encodes SEQ ID NO:2, was codon optimized for expression in E. coli and the codon-optimized polynucleotide cloned into an expression vector, placing the expression of the ketoreductase gene under the control of the T7 promoter. The T7 polymerase needed to express the gene of interest is under control of the lac promoter, and both the gene of interest and the T7 polymerase are subject to lac1 repression. The presence of IPTG activates the T7 polymerase production and eliminates the repression, resulting in expression of the ketoreductase protein. Clones expressing the active ketoreductase in E. co/i were identified and the genes sequenced to confirm their identity.

The ketoreductases of the disclosure may be obtained by subjecting the polynucleotide encoding the parent sequence to mutagenesis and/or directed evolution methods. An exemplary directed evolution technique is mutagenesis and/or DNA shuffling as described in Stemmer, 1994, Proc. Natl. Acad. Sci. USA 91:10747-10751; WO 95/22625; WO 97/20078; WO 97/35966; WO 98/27230; WO 00/42651; WO 01/75767 and U.S. Pat. No. 6,537,746. Other directed evolution procedures that can be used include, among others, staggered extension process (StEP), in vitro recombination (Zhao et al., 1998, Nat. Biotechnol. 16:258-261), mutagenic PCR (Caldwell et al., 1994, PCR Methods Appl. 3: S136-S140), and cassette mutagenesis (Black et al., 1996, Proc. Natl. Acad. Sci. USA 93:3525-3529).

The clones obtained following mutagenesis treatment are screened for ketoreductases having a desired improved enzyme property. Measuring enzyme activity from the expression libraries can be performed using standard chemistry analytical techniques for measuring substrates and products such as UPLC-MS, as well as the standard biochemistry technique of monitoring the rate of decrease (via a decrease in absorbance or fluorescence) of NADH or NADPH concentration, as it is converted into NAD+ or NADP+. In this reaction, the NADH or NADPH is consumed (oxidized) by the ketoreductase as the ketoreductase reduces a ketone substrate to the corresponding hydroxyl group. The rate of decrease of NADH or NADPH concentration, as measured by the decrease in absorbance or fluorescence, per unit time indicates the relative (enzymatic) activity of the ketoreductase polypeptide in a fixed amount of the lysate (or a lyophilized powder made therefrom). Where the improved enzyme property desired is thermal stability, enzyme activity may be measured after subjecting the enzyme preparations to a defined temperature and measuring the amount of enzyme activity remaining after heat treatments. Clones containing a polynucleotide encoding a ketoreductase are then isolated, sequenced to identify the nucleotide sequence changes (if any), and used to express the enzyme in a host cell.

Where the sequence of the polypeptide is known, the polynucleotides encoding the enzyme can be prepared by standard solid-phase methods, according to known synthetic methods. In some embodiments, fragments of up to about 100 bases can be individually synthesized, then joined (e.g., by enzymatic or chemical litigation methods, or polymerase mediated methods) to form any desired continuous sequence. For example, polynucleotides and oligonucleotides of the invention can be prepared by chemical synthesis using, e.g., the classical phosphoramidite method described by Beaucage et al., 1981, Tet. Lett. 22:1859-69, or the method described by Matthes et al., 1984, EMBO J. 3:801-05, e.g., as it is typically practiced in automated synthetic methods. According to the phosphoramidite method, oligonucleotides are synthesized, e.g., in an automatic DNA synthesizer, purified, annealed, ligated and cloned in appropriate vectors. In addition, essentially any nucleic acid can be obtained from any of a variety of commercial sources, such as The Midland Certified Reagent Company, Midland, Tex., The Great American Gene Company, Ramona, Calif, ExpressGen Inc. Chicago, Ill., Operon Technologies Inc., Alameda, Calif., and many others.

Ketoreductase enzymes expressed in a host cell can be recovered from the cells and or the culture medium using any one or more of the well-known techniques for protein purification, including, among others, lysozyme treatment, sonication, filtration, salting-out, ultra-centrifugation, and chromatography. Suitable solutions for lysing and the high efficiency extraction of proteins from bacteria, such as E. coli, are commercially available under the trade name CelLytic B® from Sigma-Aldrich of St. Louis Mo.

Chromatographic techniques for isolation of the ketoreductase polypeptide include, among others, reverse phase chromatography high performance liquid chromatography, ion exchange chromatography, gel electrophoresis, and affinity chromatography. Conditions for purifying a particular enzyme will depend, in part, on factors such as net charge, hydrophobicity, hydrophilicity, molecular weight, molecular shape, etc., and will be apparent to those having skill in the art.

In some embodiments, affinity techniques may be used to isolate the improved ketoreductase enzymes. For affinity chromatography purification, the protein sequence can be tagged with a recognition sequence to enable purification. Common tags include celluose-binding domains, poly His-tags, di-His chelates, FLAG-tags and many others that will be apparent to those having skill in the art. Antibodies can also be used as affinity purification reagents. Any antibody that specifically binds the ketoreductase polypeptide may be used.

### Processes for Using the Ketoreductases

The ketoreductase enzymes described herein can catalyze the reduction of substrate substituted indanone compounds such as fluoro hydroxyindanone (6)

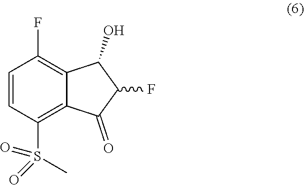 [SMILES: CS(=O)(=O)c1ccc(F)c2c1C(=O)C(F)[C@H]2O]

to the fluorodiol (7)

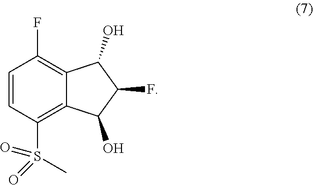 [SMILES: CS(=O)(=O)c1ccc(F)c2c1[C@H](O)[C@H](F)[C@H]2O]

In particular embodiments, the ketoreductase enzymes described herein can be used immediately following electrophilic fluorination of hydroxyindanone (5):

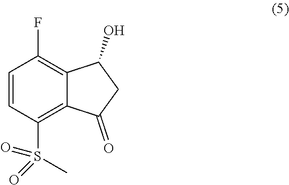 [SMILES: CS(=O)(=O)c1ccc(F)c2c1C(=O)C[C@H]2O]

to provide fluorodiol (7).

In some embodiments, the process for preparing fluorodiol (7) comprises contacting hydroxy indanone (5) with a fluorinating agent under acidic conditions to yield fluoro hydroxyindanone (6); contacting the fluoro hydroxyindanone (6) with a ketoreductase disclosed herein under reaction conditions suitable for reducing or converting fluoro hydroxyindanone (6) to fluorodiol (7). Fluorodiol (7) is an intermediate for the synthesis of belzutifan (WELIREG). Thus, in a process for preparing belzutifan, the process can comprise a step in which hydroxy indanone (5) is converted to fluorodiol (7) using a ketoreductase disclosed herein. In some embodiments, the product in greater than about 99%, 99.1%, 99.2%, 99.3%, 99.4%, 99.5%, 99.6%, 60 99.7%, 99.8%, or 99.9% diastereometric excess over the corresponding (1R) alcohol product.

As noted herein, in some embodiments, the ketoreductases can comprise an amino acid sequence that is at least about 85%, 86%, 87%, 88%, 89%, 90%, 91%, 92%, 93%, 94%, 95%, 96%, 97%, 98%, or 99% identical as compared a reference sequence comprising the sequence of SEQ ID NO: 2. In some embodiments, these ketoreductase polypeptides can have one or more modifications to the amino acid sequence of SEQ ID NO: 2. The modifications can include substitutions, deletions, and insertions. The substitutions can be non-conservative substitutions, conservative substitutions, or a combination of nonconservative and conservative substitutions.

In some embodiments of the method for reducing the substrate to the product, the substrate is reduced to the product in greater than about 99% stereometric excess, wherein the ketoreductase polypeptide comprises a sequence that corresponds to SEQ ID NO: 2.

In another embodiment of this method for reducing the substrate to the product, at least about 95% of the substrate is converted to the product in less than about 24 hours when carried out with greater than about 60 g/L of substrate and less than about 5 g/L of the polypeptide, wherein the polypeptide comprises an amino acid sequence corresponding to SEQ ID NO: 2.

As is known by those of skill in the art, ketoreductase catalyzed reduction reactions typically require a cofactor. Reduction reactions catalyzed by the ketoreductase enzymes described herein also typically require a cofactor, although many embodiments of the engineered ketoreductases require far less cofactor than reactions catalyzed with wild-type ketoreductase enzymes. As used herein, the term “cofactor” refers to a non-protein compound that operates in combination with a ketoreductase enzyme. Cofactors suitable for use with the engineered ketoreductase enzymes described herein include, but are not limited to, NADP+(nicotinamide adenine dinucleotide phosphate), NADPH (the reduced form of NADP+), NAD+(nicotinamide adenine dinucleotide) and NADH (the reduced form of NAD+). Generally, the reduced form of the cofactor is added to the reaction mixture. The reduced NAD(P)H form can be optionally regenerated from the oxidized NAD(P)+ form using a cofactor regeneration system.

The term “cofactor regeneration system” refers to a set of reactants that participate in a reaction that reduces the oxidized form of the cofactor (e.g., NADP+ to NADPH). Cofactors oxidized by the ketoreductase-catalyzed reduction of the keto substrate are regenerated in reduced form by the cofactor regeneration system. Cofactor regeneration systems comprise a stoichiometric reductant that is a source of reducing hydrogen equivalents and that is capable of reducing the oxidized form of the cofactor. The cofactor regeneration system may further comprise a catalyst, for example an enzyme catalyst that catalyzes the reduction of the oxidized form of the cofactor by the reductant. In some embodiments, the ketoreductase of the disclosure itself may serve as the enzyme catalyst that catalyzes the reduction of the oxidized form of the cofactor by the reductant. Thus, in such embodiments, the ketoreductase serves a dual catalytic role. Cofactor regeneration systems to regenerate NADH or NADPH from NAD+ or NADP+, respectively, are known in the art and may be used in the methods described herein.

Suitable exemplary cofactor regeneration systems that may be employed include, but are not limited to, glucose and glucose dehydrogenase, formate and formate dehydrogenase, glucose-6-phosphate and glucose-6-phosphate dehydrogenase, a secondary (e.g., isopropanol) alcohol and secondary alcohol dehydrogenase, phosphite and phosphite dehydrogenase, molecular hydrogen and hydrogenase, and the like. These systems may be used in combination with either NADP+/NADPH or NAD+/NADH as the cofactor. Electrochemical regeneration using hydrogenase may also be used as a cofactor regeneration system. See e.g., U.S. Pat. Nos. 5,538,867 and 6,495,023, both of which are incorporated herein by reference. Chemical cofactor regeneration systems comprising a metal catalyst and a reducing agent (for example, molecular hydrogen or formate) are also suitable. See e.g., PCT publication WO 2000/053731, which is incorporated herein by reference.

The terms “glucose dehydrogenase” and “GDH” are used interchangeably herein to refer to an NAD+ or NADP+-dependent enzyme that catalyzes the conversion of D-glucose and NAD+ or NADP+ to gluconic acid and NADH or NADPH, respectively.

Glucose dehydrogenases that are suitable for use in the practice of the methods described herein include both naturally occurring glucose dehydrogenases, as well as nonnaturally occurring glucose dehydrogenases. Naturally occurring glucose dehydrogenase encoding genes have been reported in the literature. For example, the Bacillus subtilis 61297 GDH gene was expressed in E. coli and was reported to exhibit the same physicochemical properties as the enzyme produced in its native host (Vasantha et al., 1983, Proc. Natl. Acad. Sci. USA 80:785). The gene sequence of the B. subtilis GDH gene, which corresponds to Genbank Acc. No. M12276, was reported by Lampel et al., 1986, J. Bacterial. 166:238-243, and in corrected form by Yamane et al., 1996, Microbiology 142:3047-3056 as Genbank Acc. No. D50453. Naturally occurring GDH genes also include those that encode the GDH from B. cereus ATCC 14579 (Nature, 2003, 423:87-91; Genbank Acc. No. AE017013) and B. megaterium (Eur. J. Biochem., 1988, 174:485-490, Genbank Acc. No. X12370; J. Ferment. Bioeng., 1990, 70:363-369, Genbank Acc. No. GI216270). Glucose dehydrogenases from Bacillus sp. are provided in PCT publication WO 2005/018579, the disclosure of which is incorporated herein by reference.

Non-naturally occurring glucose dehydrogenases may be generated using known methods, such as, for example, mutagenesis, directed evolution, and the like. GDH enzymes having suitable activity, whether naturally occurring or non-naturally occurring, may be readily identified by one of ordinary skill in the art, including using the assay described in Example 4 of PCT publication WO 2005/018579, the disclosure of which is incorporated herein by reference.

The ketoreductase-catalyzed reduction reactions described herein are generally carried out in a solvent. Suitable solvents include water, organic solvents, (e.g., ethyl acetate, butyl acetate, 1-octanol, heptane, octane, methyl t-butyl ether (MTBE), toluene, and the like), and ionic liquids (e.g., 1-ethyl 4-methylimidazolium tetrafluoroborate, l-butyl-3-methylimidazolium tetrafluoroborate, 1-butyl-3-methylimidazolium hexafluorophosphate, and the like). In some embodiments, aqueous solvents, including water and aqueous co-solvent systems, are used.

Exemplary aqueous co-solvent systems have water and one or more organic solvent. In general, an organic solvent component of an aqueous co-solvent system is selected such that it does not completely inactivate the ketoreductase enzyme. Appropriate co-solvent systems can be readily identified by measuring the enzymatic activity of the specified engineered ketoreductase enzyme with a defined substrate of interest in the candidate solvent system, utilizing an enzyme activity assay, such as those described herein.

The organic solvent component of an aqueous co-solvent system may be miscible with the aqueous component, providing a single liquid phase, or may be partly miscible or immiscible with the aqueous component, providing two liquid phases. Generally, when an aqueous co-solvent system is employed, it is selected to be biphasic, with water dispersed in an organic solvent, or vice-versa. Generally, when an aqueous co-solvent system is utilized, it is desirable to select an organic solvent that can be readily separated from the aqueous phase. In general, the ratio of water to organic solvent in the co-solvent system is typically in the range of from about 90:10 to about 10:90 (v/v) organic solvent to water, and between 80:20 and 20:80 (v/v) organic solvent to water. The co-solvent system may be pre-formed prior to addition to the reaction mixture, or it may be formed in situ in the reaction vessel.

The aqueous solvent (water or aqueous co-solvent system) may be pH-buffered or unbuffered. Generally, the reduction can be carried out at a pH of about 10 or below, usually in the range of from about 5 to about 10. In some embodiments, the reduction is carried out at a pH of about 9 or below, usually in the range of from about 5 to about 9. In some embodiments, the reduction is carried out at a pH of about 8 or below, often in the range of from about 5 to about 8, and usually in the range of from about 6 to about 8. The reduction may also be carried out at a pH of about 7.8 or below, or 7.5 or below. Alternatively, the reduction may be carried out a neutral pH, i.e., about 7.

During the course of the reduction reactions, the pH of the reaction mixture may change. The pH of the reaction mixture may be maintained at a desired pH or within a desired pH range by the addition of an acid or a base during the course of the reaction. Alternatively, the pH may be controlled by using an aqueous solvent that comprises a buffer. Suitable buffers to maintain desired pH ranges are known in the art and include, for example, phosphate buffer, triethanolamine buffer, and the like. Combinations of buffering and acid or base addition may also be used.

When the glucose/glucose dehydrogenase cofactor regeneration system is employed, the co-production of gluconic acid (pKa=3.6), as represented in equation (1) causes the pH of the reaction mixture to drop if the resulting aqueous gluconic acid is not otherwise neutralized. The pH of the reaction mixture may be maintained at the desired level by standard buffering techniques, wherein the buffer neutralizes the gluconic acid up to the buffering capacity provided, or by the addition of a base concurrent with the course of the conversion. Combinations of buffering and base addition may also be used. Suitable buffers to maintain desired pH ranges are described above. Suitable bases for neutralization of gluconic acid are organic bases, for example amines, alkoxides and the like, and inorganic bases, for example, hydroxide salts (e.g., NaOH), carbonate salts (e.g., NaHCO₃), bicarbonate salts (e.g., K₂CO₃), basic phosphate salts (e.g., K₂HPO₄, Na₃PO₄), and the like. The addition of a base concurrent with the course of the conversion may be done manually while monitoring the reaction mixture pH or, more conveniently, by using an automatic titrator as a pH stat. A combination of partial buffering capacity and base addition can also be used for process control.

When base addition is employed to neutralize gluconic acid released during a ketoreductase-catalyzed reduction reaction, the progress of the conversion may be monitored by the amount of base added to maintain the pH. Typically, bases added to unbuffered or partially buffered reaction mixtures over the course of the reduction are added in aqueous solutions.

In some embodiments, the co-factor regenerating system can comprises a formate dehydrogenase. The terms “formate dehydrogenase” and “FDH” are used interchangeably herein to refer to an NAD+ or NADP+-dependent enzyme that catalyzes the conversion of formate and NAD+ or NADP+ to carbon dioxide and NADH or NADPH, respectively. Formate dehydrogenases that are suitable for use as cofactor regenerating systems in the ketoreductase-catalyzed reduction reactions described herein include both naturally occurring formate dehydrogenases, as well as non-naturally occurring formate dehydrogenases. Formate dehydrogenases include those corresponding to SEQ ID NOS: 70 (Pseudomonas sp.) and 72 (Candida boidinii) of PCT publication WO 2005/018579, which are encoded by polynucleotide sequences corresponding to SEQ ID NOS: 69 and 71, respectively, of PCT publication WO 2005/018579, the disclosures of which are incorporated herein by reference. Formate dehydrogenases employed in the methods described herein, whether naturally occurring or non-naturally occurring, may exhibit an activity of at least about 1 μmol/min/mg, sometimes at least about 10 μmol/min/mg, or at least about 10² μmol/min/mg, up to about 10³ μmol/min/mg or higher, and can be readily screened for activity in the assay described in Example 4 of PCT publication WO 2005/018579.

As used herein, the term “formate” refers to formate anion (HCO₂⁻), formic acid (HCO₂H), and mixtures thereof. Formate may be provided in the form of a salt, typically an alkali or ammonium salt (for example, HCO₂Na, KHCO₂NH₄, and the like), in the form of formic acid, typically aqueous formic acid, or mixtures thereof. Formic acid is a moderate acid. In aqueous solutions within several pH units of its pKa (pKa=3.7 in water) formate is present as both HCO₂⁻ and HCO₂H in equilibrium concentrations. At pH values above about pH 4, formate is predominantly present as HCO₂⁻. When formate is provided as formic acid, the reaction mixture is typically buffered or made less acidic by adding a base to provide the desired pH, typically of about pH 5 or above. Suitable bases for neutralization of formic acid include, but are not limited to, organic bases, for example amines, alkoxides and the like, and inorganic bases. When the glucose/glucose dehydrogenase cofactor regeneration system is employed, the co-production of gluconic acid (pKa=3.6) causes the pH of the reaction mixture to drop if the resulting aqueous gluconic acid is not otherwise neutralized. The pH of the reaction mixture may be maintained at the desired level by standard buffering techniques, wherein the buffer neutralizes the gluconic acid up to the buffering capacity provided, or by the addition of a base concurrent with the course of the conversion. Combinations of buffering and base addition may also be used. Suitable buffers to maintain desired pH ranges are described above. Suitable bases for neutralization for example, hydroxide salts (e.g., NaOH), carbonate salts (e.g., NaHCO₃), bicarbonate salts (e.g., K₂CO₃), basic phosphate salts (e.g., K₂HPO₄, Na₃PO₄), and the like.

When formate and formate dehydrogenase are employed as the cofactor regeneration system, the pH of the reaction mixture may be maintained at the desired level by standard buffering techniques, wherein the buffer releases protons up to the buffering capacity provided, or by the addition of an acid concurrent with the course of the conversion. Suitable acids to add during the course of the reaction to maintain the pH include organic acids, for example carboxylic acids, sulfonic acids, phosphonic acids, and the like, mineral acids, for example hydrohalic acids (such as hydrochloric acid), sulfuric acid, phosphoric acid, and the like, acidic salts, for example dihydrogenphosphate salts (e.g., KH₂PO₄), bisulfate salts (e.g., NaHSO₄) and the like. Some embodiments utilize formic acid, whereby both the formate concentration and the pH of the solution are maintained.

When acid addition is employed to maintain the pH during a reduction reaction using the formate/formate dehydrogenase cofactor regeneration system, the progress of the conversion may be monitored by the amount of acid added to maintain the pH. Typically, acids added to unbuffered or partially buffered reaction mixtures over the course of conversion are added in aqueous solutions.

In carrying out embodiments of the ketoreductase-catalyzed reduction reactions described herein employing a cofactor regeneration system, either the oxidized or reduced form of the cofactor may be provided initially. As described above, the cofactor regeneration system converts oxidized cofactor to its reduced form, which is then utilized in the reduction of the ketoreductase substrate.

In some embodiments, cofactor regeneration systems are not used. For reduction reactions carried out without the use of a cofactor regenerating systems, the cofactor is added to the reaction mixture in reduced form.

In some embodiments, when the process is carried out using whole cells of the host organism, the whole cell may natively provide the cofactor. Alternatively or in combination, the cell may natively or recombinantly provide the glucose dehydrogenase.

In carrying out the stereoselective reduction reactions described herein, the ketoreductase enzyme, and any enzymes comprising the optional cofactor regeneration system, may be added to the reaction mixture in the form of the purified enzymes, whole cells transformed with gene(s) encoding the enzymes, and/or cell extracts and/or lysates of such cells. The gene(s) encoding the ketoreductase enzyme and the optional cofactor regeneration enzymes can be transformed into host cells separately or together into the same host cell. For example, in some embodiments one set of host cells can be transformed with gene(s) encoding the ketoreductase enzyme and another set can be transformed with gene(s) encoding the cofactor regeneration enzymes. Both sets of transformed cells can be utilized together in the reaction mixture in the form of whole cells, or in the form of lysates or extracts derived therefrom. In other embodiments, a host cell can be transformed with gene(s) encoding both the ketoreductase enzyme and the cofactor regeneration enzymes.

Whole cells transformed with gene(s) encoding the ketoreductase enzyme and/or the optional cofactor regeneration enzymes, or cell extracts and/or lysates thereof, may be employed in a variety of different forms, including solid (e.g., lyophilized, spray-dried, and the like) or semisolid (e.g., a crude paste).

The cell extracts or cell lysates may be partially purified by precipitation (ammonium sulfate, polyethyleneimine, heat treatment or the like), followed by a desalting procedure prior to lyophilization (e.g., ultrafiltration, dialysis, and the like). Any of the cell preparations may be stabilized by crosslinking using known crosslinking agents.

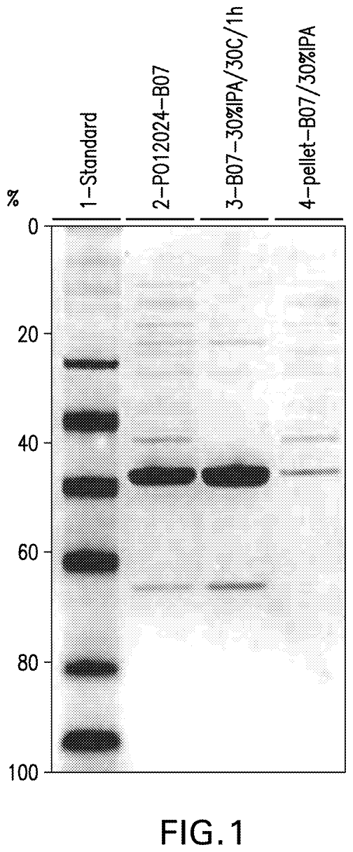

In some embodiments, ketoreductase enzymes with improved purity may be desired. In such embodiments, clarified cell lysates used to obtain the ketoreductase enzymes may be pre-treated with isopropanol, to bring the volume % of isopropanol to 25%-30%. The isopropanol treated lysates may be incubated for between 1 hour and overnight at 30° C. The isopropanol-treated lysate can then be centrifuged, and the pellet may be removed. The supernatant may then be transferred to a petri dish and frozen at −80° C. for a minimum of 2 hours. Samples may then be lyophilized using a standard automated protocol. As showing in FIG. 1, gel electrophoresis, using sodium dodecyl sulfate (SDS, also known as sodium lauryl sulfate) and polyacrylamide gel (also known as SDS-PAGE), shows the removal of proteins that are insoluble in isopropyl alcohol (iPrOH), providing purified ketoreductase. In FIG. 1, lane 1 (indicated by Standard) is the marker; lane 2 (indicated by P012024-B07) is the crude enzyme preparation; lane 3 (indicated by B07, 30% IPA/30% IPA/30C/1 h) is the crude enzyme preparation preparation treated with a solution of 30% iPrOH for 1 hr and centrifuged; and lane 4 (indicated by 4 pellet-B07/30% IPA) are the solids from the centrifugation. As is seen in FIG. 1, lane 3 shows the removal of many bands from the enzyme preparation upon treatment with iPrOH. Lane 4 shows the proteins that proteins were precipitated and remove by centrifugation.

Suitable conditions for carrying out the ketoreductase catalyzed reduction reactions described herein include a wide variety of conditions that can be readily optimized by routine experimentation that includes, but is not limited to, contacting the engineered ketoreductase enzyme and substrate at an experimental pH and temperature and detecting product, for example, using the methods described in the Examples provided herein.

The ketoreductase catalyzed reduction is typically carried out at a temperature in the range of from about 15° C. to about 75° C. For some embodiments, the reaction is carried out at a temperature in the range of from about 20° C. to about 55° C. In still other embodiments, it is carried out at a temperature in the range of from about 20° C. to about 45° C. The reaction may also be carried out under ambient conditions.

The reduction reaction is generally allowed to proceed until essentially complete, or near complete, reduction of substrate is obtained. Reduction of substrate to product can be monitored using known methods by detecting substrate and/or product. Suitable methods include gas chromatography, HPLC, and the like. Conversion yields of the alcohol reduction product generated in the reaction mixture are generally greater than about 50%, may also be greater than about 60%, may also be greater than about 70%, may also be greater than about 80%, may also be greater than 90%, and are often greater than about 97%.

### EXAMPLES

### Example 1: Enzyme Preparation

E. coli cultures, each harboring a plasmid that encodes a ketoreductase enzyme that can be represented by amino acid sequence as set forth below in SEQ ID NO. 1, 2, 4-16 as set forth above, were diluted serially to dilutions of 10⁻⁴, 10⁻⁵, and 10⁻⁶ using Luria-Bertani Broth (culture media for cells) as a diluent. 100 μL of the dilutions were each spread on a petri dish containing LB ager, supplemented with 50 μg/mL of Kanamycin. The plates were placed in a 30° C. incubator overnight.

200 μL per well of Luria-Bertani Broth (culture media for cells) (500 mL LB+50 μg/mL Kanamycin) was aliquoted into labeled 96-well shallow well plates. The shallow well plates were loaded into a plate stacker of a colony picker. Agar plates containing colonies sufficiently diluted so that the majority of colonies were isolated from one another (known as single colonies to those schooled in the art) were picked into unique wells of the shallow well plates. The colonies were allowed to grow overnight, at 200 rpm, at 30° C., and 85% RH.

390 μL of Terrific Broth (TB) growth media (commercially available from ThermoFisher Scientific as Catalog #A1374301) (TB+50 μg/mL Kanamycin) was aliquoted into labeled 96-well deep well subculture plates. 13 μL of the overnight growth culture was transferred from each well of the master shallow well plate into the corresponding labeled deep well subculture plates. The plates were sealed with breathable film, and the plates were shaken for 2-2.5 h at 250 rpm, at 30° C. and 85% RH. After shaking, optical density (OD₆₀₀, optical density at 600 nm wavelength) of at least one plate was measured for growth. When the OD₆₀₀ of this plate was in the range of 0.4-0.8, the deep well plates were induced with 4 μL per well of 1 M IPTG solution. The plates were resealed and incubated with shaking for 18-20 h at 250 rpm, at 30° C., and 85% RH.

After incubation, all deep well plates were centrifuged for 15 min. at 4000 rpm at 4° C. Following centrifugation, the supernatant was discarded. The plates were heat-sealed and stored at −80° C.

The cell plates were removed from −80° C. storage and defrosted at RT. A lysis buffer of 100 mM potassium phosphate pH 8.0, 1 mg/mL lysozyme, 0.50 mg/mL polymyxin B sulfate (PMBS), 3 units/mL DNase I, 4 mM MgCl₂ and 1 mg/mL NADP⁺ was prepared. 200 μL of the lysis buffer was aliquoted into each well. The lysis mixture was shaken for 1-1.5 h at 1000 rpm on a plate shaker at RT. The lysis mixture was then centrifuged for 15 min at 4000 rpm at 4° C. to make the enzyme containing lysate solution (which is in the supernatant). In some instances, the lysate solution was further incubated with isopropanol in solutions of 25-30% isopropanol for 1-5 h. After incubation, the lysate was centrifuged as before.

### Example 2: Ketoreductase Reaction in Well Plates

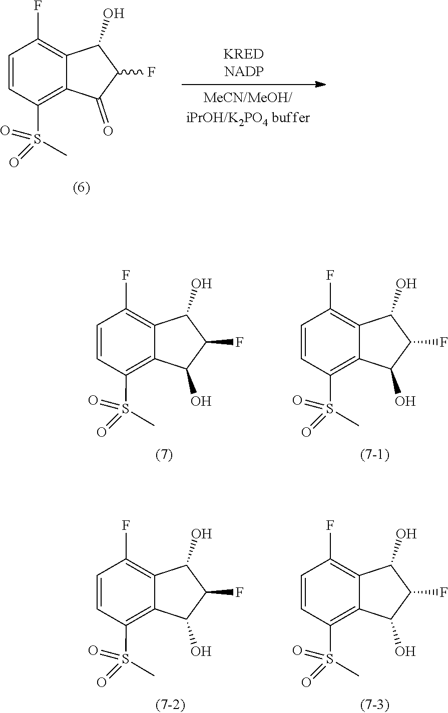 [SMILES: CS(=O)(=O)c1ccc(F)c2c1C(=O)C(F)[C@H]2O ; CS(=O)(=O)c1ccc(F)c2c1[C@H](O)[C@H](F)[C@H]2O ; CS(=O)(=O)c1ccc(F)c2c1[C@H](O)[C@@H](F)[C@H]2O ; CS(=O)(=O)c1ccc(F)c2c1[C@@H](O)[C@H](F)[C@H]2O ; CS(=O)(=O)c1ccc(F)c2c1[C@@H](O)[C@@H](F)[C@H]2O]

A reaction buffer was prepared by resuspending the solid stream substrate (an impure product (6) of the previous chemical fluorination reaction, see Example 5 below) to a concentration of 75-100 g/L in a 16:16:10:58 (v/v) acetonitrile:methanol:isopropanol:potassium phosphate buffer pH 8.0 solution. The pH was then further adjusted to 8.0 using aqueous sodium hydroxide.

80 μL of the reaction buffer was added to 0.3 mL round bottom well plates, followed by 20 μL of the enzyme-containing lysate solution of Example 1. The plate was heat sealed and shaken overnight at 35° C. and 1000 rpm.

After overnight shaking, 30 μL from the reaction was added to a new round bottom well plate containing 240 μL of acetonitrile. This mixture was allowed to age for one hour, at which point 30 μL was added to the top of a filter stack (filter plates on top of the round bottom plates, with 0.20 μM hydrophilic PTFE, commercially available from Millipore MSRLN2250) containing an additional 240 μL of 20% (v/v) acetonitrile in water. The plates were then centrifuged for 3 min at 4000 rpm at 4° C. The filter plates were removed, and the clear solutions in the receiving plates were heat sealed.

The filtered solutions were analyzed by ultra-performance liquid chromatography (UPLC), using a high throughput screening method to monitor the substrate and degradation of the substrates, and the product peak areas. UPLC was conducted on an Agilent instrument with a Waters HSS T3 1.8 μm, 2.1×75 mm column using an isocratic method of 14% CH₃CN+0.1% TFA/H₂O+0.1% TFA over 1.1 min; flow rate 1 mL/min. The two diastereomers of the starting material eluted at 0.53 min and 0.66 min, the desired product diastereomer (7) at 0.46 min and the undesired product diastereomers (7-1, 7-2, 7-3) at 0.48 min, 0.88 min, and 0.47 min, respectively. The diastereoselectivity was determined through supercritical fluid chromatography with an Agilent instrument on a Daicel CHIRALPAK IG-3 3.0 μm, 50×4.6 mm column. Mobile phase A was supercritical CO₂, mobile phase B was isopropanol. Flow rate was 2.5 mL/min; linear gradient 18-37% B over 1.5 min, 37% B for 0.2 min, linear gradent to 18% B over 0.05 min and 0.15 min equilibration time at 18% B (total time 1.9 min). The two diastereomers of the starting material eluted at 0.79 min and 1.6 min, the desired product diastereomer (7) at 1.1 min, and the undesired product diastereomers (7-1, 7-2, 7-3) eluted at 1.3 min, 0.9 min and 0.6 min, respectively.

### Example 3: Enzyme Preparation in Shaker Flasks

5 mL of Luria-Bertani Broth (culture media for cells) (250 mL LB+50 μg/ml Kanamycin+1% glucose) was inoculated with 10 μL of E. coli cells, each harboring a plasmid that encodes a ketoreductase enzyme that can be represented by amino acid sequence as set forth below in SEQ ID NO. 1, 2, and 4-16, as set forth below, that had been stored at −80° C. in 20% glycerol and aliquoted into labeled 15 mL cell culture tubes. The cell culture tubes were sealed and incubated with shaking for 20-24 h at 250 rpm, at 30° C.

After overnight growth, the overnight growth cultures (2-5 mL of cell culture (having a starting OD₆₀₀ of 0.2)) were added to 250 mL of Terrific Broth (TB) growth media (commercially available from ThermoFisher Scientific as Catalog #A1374301) (TB+50 μg/ml Kanamycin) to a final volume of 250 mL. The flasks were shaken for 3-4 h at 250 rpm, at 30° C. After shaking, OD₆₀₀ was measured for growth until OD₆₀₀ reached 0.4-0.6. At this point, 1 mM of IPTG (250 μL of 1 M IPTG) was added to the culture to induce expression, and the culture was allowed to grow 20-24 h at 250 rpm, at 30° C.

After the additional growth period, the cultures were transferred to a centrifuge bottle of known weight and centrifuged for 20 min. at 4000 rpm at 4° C. Following centrifugation, the supernatant was discarded, and the remaining cell pellet in the bottle was weighed. The weight of the cell pellet was calculated by subtracting the known weight of the bottle, and the cell pellet was resuspended in 5 volumes of 50 mM sodium phosphate buffer (pH=7), at a volume that is 5 times the weight of the cell pellet.

The cells from the resuspended cell pellets were lysed using a microfluidizer, and the cell lysate was collected and centrifuged for 60 min. at 10000 rpm at 4° C. The clarified lysate was occasionally further treated with isopropanol in solutions of 25-30% isopropanol for 1-5 h. After incubation, the lysate was centrifuged as before. The clarified supernatant was transferred to a petri dish and frozen at −80° C. for approximately 2 h. Samples were lyophilized using a standard automated protocol.

### Example 4: Ketoreductase Reaction

To a 60 mL aqueous solution of 200 mM K₂HPO₄ was dissolved 0.90 g of NADP⁺, followed by 2.4 g of enzyme powder as prepared in Example 3. 135 mL of isopropanol was added to the solution and the pH was adjusted to 8.0 with aqueous 5 N NaOH. The above enzyme solution was added to the quenched chemical fluorination reaction mixture containing 60 g of the fluoroketone substrate (6). The temperature was set at 33° C., and the reaction mixture was stirred for 18 h. The enzymatic reaction mixture was quenched with 900 mL of ethyl acetate, cooled to 20° C. and filtered. The biphasic mixture resulting from the filtrate was separated and the organic layer was washed with 40% (w/v) aqueous (NH₄)₂SO₄ (2×240 mL), then 25% (w/v) aqueous K₂HPO₄ (2×120 mL). The volume of the organics was reduced to 900 mL by distillation and mixed with 500 mL of ethyl acetate. The volume was reduced again to 900 mL, mixed with 200 mL of ethyl acetate, and concentrated a third time to 900 mL by distillation. CUNO #5 (7.8 g) was added to the saturated solution and the mixture stirred at room temperature for 45 min before filtration through CELITE. The filtrate was distilled to 600 mL at 60° C. then cooled to 30° C. over 45 min. The supersaturated solution was stirred at 350 rpm and seeded with product crystals (600 mg). Toluene (518 mL) was added over 4 h and the mixture was distilled to 360 mL at 29-33° C. over 1.5 h, then cooled to 20° C. over 2 h. After 14 h of aging at 20° C., the slurry was filtered and the solids were washed with 120 mL of 1:4 (v/v) ethyl acetate:toluene then 120 mL of toluene. The wet cake was placed in a 45° C. vacuum to dry for 1 day to provide the fluorodiol (7).

### Example 5: Preparation of (1S,2R,3S)-2,4-difluoro-7-(methylsulfonyl)-2,3-dihydro-1H-indene-1,3-diol (7)

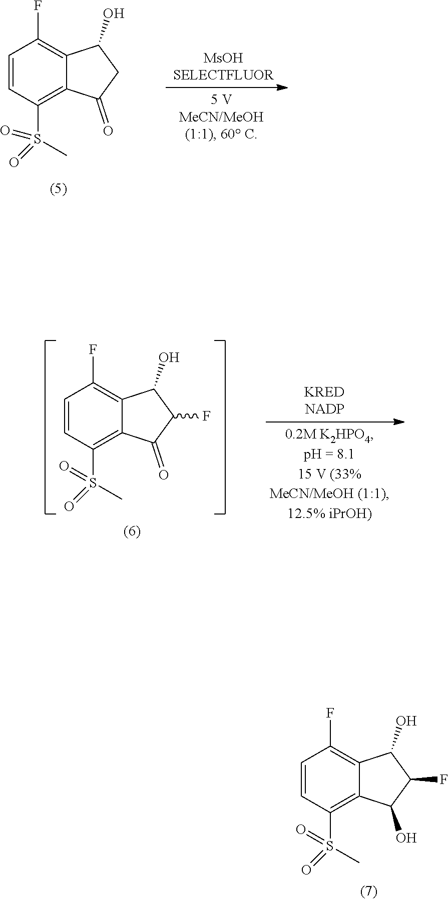

Acetonitrile (2.5 L), methanol (2.5 L), (5) (1.0 kg, 1.0 equiv), methane sulfonic acid (118 g, 0.3 equiv), and SELECTFLUOR (1.596 kg, 1.1 equiv) were charged to a five-gallon Hastelloy C-276 reactor. The vessel was pressure purged with N₂ a total of five times, and then the resulting mixture was agitated and heated to 60° C. for 16 h. Methyl acetoacetate (95 g, 0.2 equiv) and water (369 g, 5 equiv) were charged and the batch was further aged at 60° C. for 2 h. The batch was then cooled to 20° C. and 0.2 M K₂HPO₄ (7.75 L) was added. 50 wt % NaOH (344 g, 1.05 equiv) was added over 15 min to neutralize the acidic solution (final pH=6.3). This mixture was agitated and then transferred into high density polyethylene carboys. The reaction mixture was then vacuum transferred into a 30 L glass reactor. Isopropanol (2.25 L) was added to the reaction followed by a solution containing KRED and NADP (1.5 L 0.2 M K₂HPO₄ containing 40 g KRED and 15 g NADP). The solution pH was adjusted by adding 5 N NaOH (288 g, 0.3 equiv) over 25 minutes (final pH=8.1). The solution was agitated and heated to 34° C. for 17 h. The reaction volume was reduced to 10 L via batch concentration. When the batch volume reached 10 L, D.I. H₂O (1 L) was added and the batch was concentrated to 10 L total volume. K₂CO₃-treated CELITE (44 g, 0.044 w/w) and (NH₄)2SO₄ (5.8 kg, 5.8 w/w) were charged to the reactor and the batch was agitated and heated to 50° C. for 2 h. EtOAc (15 L) was charged at 50° C. and mixed for 30 min. The reaction was then cooled to 20° C. and aged for 30 min. The slurry was filtered through a 2′ filter pot lined with polypropylene and filter paper and the waste cake was washed with EtOAc (2×5 L). The filtrates were combined in a 50 L glass reactor and the aqueous phase was drained and discarded. The organic layer was sequentially washed with 40% (w/w) (NH₄)₂SO₄ (2×4 L), 25% (w/w) K₂HPO₄ (2 L) and 50% (w/w) K₂HPO₄ (2 L). The resulting organics were concentrated to 15 L and distilled at constant volume via addition of fresh EtOAc until <0.3 wt % water. The batch was transferred to a 50 L round bottom flask, CUNO #5 (130 g, 0.13 w/w) was added and the mixture was agitated under ambient conditions for 30 min. The slurry was filtered through a 10″ filter pot lined with polypropylene and filter paper and the filter cake was washed with EtOAc (2×2 L). The filtrate was transferred into a 20 L glass vessel and concentrated to 10 L at 60° C., then cooled to 30° C., and seeded with (7) (10 g, 0.01 w/w). The resulting slurry was aged at 30° C. for 1 h, followed by addition of toluene (8.5 L) over 4 h and further aging for 9 h. The mixture was concentrated to 6 L, then distilled at constant volume via addition of fresh toluene (2 L). The slurry was cooled 20° C. over 30 min, aged for 1 h, and filtered. The product cake was washed with 1:4 EtOAc/toluene (2 L) and toluene (2 L), and then dried in a vacuum oven at 40° C. to give (7) as an off-white solid (950 g, 85% yield, 96.7 area % by LC, 96.3 wt %). ¹H NMR (599.90 MHz, DMSO-d₆) δ 7.92 (ddd, J=8.6, 4.7 and 0.7 Hz, 1H, CH), 7.46 (t, J=8.9 Hz, 1H, CH), 6.14 (d, J=7.1 Hz, 1H, OH), 5.96 (d, J=6.9 Hz, 1H, OH), 5.56 (dd, J=6.8, 5.2 and 3.2 Hz, 1H, CH), 5.40 (ddd, J=14.1, 7.0 and 5.2 Hz, 1H, CH), 4.89 (dt, J=51.1 and 5.2 Hz, 1H, CH), 3.31 (s, 3H, CH3) ppm. 13C {¹H} NMR (150.85 MHz, DMSO-d₆) δ 162.31 (d, J_{CF}=258.7 Hz, CF), 142.85 (dd, J_{CF}=6.0 and 2.9 Hz, C), 133.89 (d, J_{CF}=3.4 Hz, C), 132.20 (d, J_{CF}=8.9 Hz, C), 130.53 (dd, J_{CF}=16.4 and 10.1 Hz, CH), 117.25 (d, J_{CF}=21.4 Hz, CH), 97.82 (d, J_{CF}=194.0 Hz, CH), 73.17 (d, J_{CF}=25.2 Hz, CH), 68.95 (d, J_{CF}=17.9 Hz, CH), 44.93 (s, CH₃) ppm. ¹⁹F NMR (564.47 MHz, DMSO-d₆) δ −111.51 (dd, J_{HF}=9.9 and 4.6 Hz, 1F), −203.88 (dd, J_{HF}=51.0 and 13.9 Hz, 1F) ppm.

SEQUENCES:

MAKIDNAVLPEGSLVLVTGANGFVGSHVVEQLLEHGYKVRGTARSASKLANLQKRWD
AKYPGRFETAVVEDMLKQGAYDEVIKGAAGVAHIASVVSFSNKYDEVVTPAIGGTLNA
LRAAAATPSVKRFVLTSSTVSALIPKPNVEGIYLDEKSWNLESIDKAKTLPESDPQKSLW
VYAASKTEAELAAWKFMDENKPHFTLNAVLPNYTIGTIFDPETQSGSTSGWMMSLENGE
VSPALALMPPQYYVSAVDIGLLHLGCLVLPQIERRRVYGTAGTFDWNTVLATFRKLYPS
KTFPADFPDQGQDLSKFDTAPSLEILKSLGRPGWRSIEESIKDLVGSETAGQTGHHHHHH
(SEQ ID NO: 1)
MKKIDNAVLPAGSLVLVTGANGFVGSHVVEQLLEHGYKVRGTARSASKLANLQKRWD
AKYPGRFETAVVEDMLKDGAYDEVIKGAAGVAHIASPVSFSPKYDEVVPPAIGGTLNAL
RAAAATPSVKRFVLTSSMMAAIVPKPNVPGIYLDEKSWNLESIDKAVTLPESHKEKGLW
VYAASKTMAEMLAWHFMDENKPHFTLNTVLPAYTIGTIFDPETQSGSTSGIVMKLENGE
VSPMLALFGPQHYVSAFDIGLLHLGCLVLPQIERRRVYGTAAPFDWNMVLAVFRKLWPS
KTFPADFPDQGQDLSVFDTRPSLEILKSLGREGWRSFEDSIKDLVGSEFDGQTGHHHHHH
(SEQ ID NO: 2)
TCGAGTTAATTAAGGCAGTGAGCGCAACGCAATTAATGTGAGTTAGCTCACTCATTA
GGCACCCCAGGCTTTACACTTTATGCTTCCGGCTCGTATGTTGTGTGGAATTGTGAGC
GGATAACAATTTCACACAGGAAACGGCTATGACCATGATTACGGATTCACTGGCCGT
CGTTTTACAATCTAGAGGCCAGCCTGGCCATAAGGAGATATACATATGAAAAAGATT
GATAACGCAGTTCTGCCGGCAGGGTCCCTGGTTCTGGTTACCGGTGCTAACGGTTTC
GTTGGTTCCCACGTGGTTGAACAGCTGCTGGAACACGGTTACAAAGTTCGTGGTACC
GCTCGTTCCGCTTCCAAACTGGCTAACCTGCAGAAACGTTGGGATGCTAAATACCCG
GGTCGTTTCGAAACCGCTGTTGTGGAAGACATGCTGAAAGATGGTGCTTACGACGAA
GTTATCAAAGGTGCTGCTGGTGTTGCTCACATCGCTTCCCCAGTGAGTTTCAGCCCGA
AATATGACGAAGTTGTTCCTCCGGCTATCGGTGGTACCCTGAACGCTCTGCGTGCTG
CTGCTGCTACCCCGTCCGTTAAACGTTTCGTTCTGACCTCCTCCATGATGGCGGCTAT
CGTGCCAAAACCGAACGTTCCGGGTATCTACCTGGACGAAAAATCCTGGAACCTGG
AATCCATCGACAAGGCTGTCACGCTGCCAGAATCCCACAAAGAAAAAGGTCTGTGG
GTATACGCTGCATCCAAGACCATGGCTGAAATGCTGGCATGGCACTTTATGGATGAG
AACAAGCCACACTTCACTCTGAACACCGTACTGCCAGCGTACACTATTGGCACTATT
TTCGATCCGGAAACTCAGTCGGGTTCCACCTCCGGTATTGTTATGAAACTGTTTAACG
GCGAGGTTTCCCCGATGCTGGCTCTGTTTGGGCCGCAGCATTACGTTTCCGCTTTCGA
TATTGGCCTGCTGCACCTGGGTTGCCTGGTTCTGCCACAAATCGAACGTCGTCGTGTT
TACGGTACGGCTGCTCCGTTCGATTGGAACATGGTTCTGGCTGTCTTCCGTAAACTGT
GGCCGTCCAAAACCTTCCCGGCTGACTTCCCAGATCAAGGTCAGGACCTGTCTGTGT
TTGACACCCGTCCGTCCCTGGAAATTCTGAAATCTCTGGGTCGCGAAGGTTGGCGTT
CCTTCGAAGACTCCATCAAAGACCTGGTTGGTAGTGAATTTGACGGCCAAACTGGCC
ACCATCACCATCACCATTAGGGAAGAGCAGATGGGCAAGCTTGACCTGTGAAGTGA
AAAATGGCGCACATTGTGCGACATTTTTTTTTGAATTCTACGTAAAAAGCCGCCGAT
ACATCGGCTGCTTTTTTTTTGATAGAGGTTCAAACTTGTGGTATAATGAAATAAGATC
ACTCCGGGGCGTATTTTTTGAGTTATCGAGATTTTCAGGAGCTAAGGAAGCTAAAAT
GGAGAAAAAAATCACTGGATATACCACCGTTGATATATCCCAATGGCATCGTAAAG
AACATTTTGAGGCATTTCAGTCAGTTGCTCAATGTACCTATAACCAGACCGTTCAGCT
GGATATTACGGCCTTTTTAAAGACCGTAAAGAAAAATAAACACAAGTTTTATCCGGC
CTTTATTCACATTCTTGCCCGCCTGATGAATGCTCATCCGGAGTTCCGTATGGCAATG
AAAGACGGTGAGCTGGTGATATGGGATAGTGTTCACCCTTGTTACACCGTTTTCCAT
GAGCAAACTGAAACGTTTTCATCGCTCTGGAGTGAATACCACGACGATTTCCGGCAG
TTTCTACACATATATTCGCAAGATGTGGCGTGTTACGGTGAAGACCTGGCCTATTTCC
CTAAAGGGTTTATTGAGAATATGTTTTTCGTCTCAGCCAATCCCTGGGTGAGTTTCAC
CAGTTTTGATTTAAACGTGGCCAATATGGACAACTTCTTCGCCCCCGTTTTCACCATG
GGCAAATATTATACGCAAGGCGACAAGGTGCTGATGCCGCTGGCGATTCAGGTTCAT
CATGCCGTTTGTGATGGCTTCCATGTCGGCAGAATGCTTAATGAATTACAACAGTAC
TGCGATGAGTGGCAGGGCGGGGCGTAACTGCAGGAGCTCAAACAGCAGCCTGTATT
CAGGCTGCTTTTTTCGTTTTGGTCTGCGCGTAATCTCTTGCTCTGAAAACGAAAAAAC
CGCCTTGCAGGGCGGTTTTTCGAAGGTTCTCTGAGCTACCAACTCTTTGAACCGAGG
TAACTGGCTTGGAGGAGCGCAGTCACCAAAACTTGTCCTTTCAGTTTAGCCTTAACC
GGCGCATGACTTCAAGACTAACTCCTCTAAATCAATTACCAGTGGCTGCTGCCAGTG
GTGCTTTTGCATGTCTTTCCGGGTTGGACTCAAGACGATAGTTACCGGATAAGGCGC
AGCGGTCGGACTGAACGGGGGGTTCGTGCATACAGTCCAGCTTGGAGCGAACTGCC
TACCCGGAACTGAGTGTCAGGCGTGGAATGAGACAAACGCGGCCATAACAGCGGAA
TGACACCGGTAAACCGAAAGGCAGGAACAGGAGAGCGCACGAGGGAGCCGCCAGG
GGGAAACGCCTGGTATCTTTATAGTCCTGTCGGGTTTCGCCACCACTGATTTGAGCGT
CAGATTTCGTGATGCTTGTCAGGGGGGCGGAGCCTATGGAAAAACGGCTTTGCCGCG
GCCCTCTCACTTCCCTGTTAAGTATCTTCCTGGCATCTTCCAGGAAATCTCCGCCCCG
TTCGTAAGCCATTTCCGCTCGCCGCAGTCGAACGACCGAGCGTAGCGAGTCAGTGAG
CGAGGAAGCGGAATATATCCTGTATCACATATTCTGCTGACGCACCGGTGCAGCCTT
TTTTCTCCTGCCACATGAAGCACTTCACTGACACCCTCATCAGTGAACCACCGCTGGT
AGCGGTGGTTTTTTTAGGCCTATGGCCTTTTTTTTTTGTGGGAAACCTTTCGCGGTAT
GGTATTAAAGCGCCCGGAAGAGAGTCAATTCAGGGTGGTGAATGTGAAACCAGTAA
CGTTATACGATGTCGCAGAGTATGCCGGTGTCTCTTATCAGACCGTTTCCCGCGTGGT
GAACCAGGCCAGCCACGTTTCTGCGAAAACGCGGGAAAAAGTGGAAGCGGCGATGG
CGGAGCTGAATTACATTCCCAACCGCGTGGCACAACAACTGGCGGGCAAACAGTCG
TTGCTGATTGGCGTTGCCACCTCCAGTCTGGCCCTGCACGCGCCGTCGCAAATTGTCG
CGGCGATTAAATCTCGCGCCGATCAACTGGGTGCCAGCGTGGTGGTGTCGATGGTAG
AACGAAGCGGCGTCGAAGCCTGTAAAGCGGCGGTGCACAATCTTCTCGCGCAACGC
GTCAGTGGGCTGATCATTAACTATCCGCTGGATGACCAGGATGCCATTGCTGTGGAA
GCTGCCTGCACTAATGTTCCGGCGTTATTTCTTGATGTCTCTGACCAGACACCCATCA
ACAGTATTATTTTCTCCCATGAAGACGGTACGCGACTGGGCGTGGAGCATCTGGTCG
CATTGGGTCACCAGCAAATCGCGCTGTTAGCGGGCCCATTAAGTTCTGTCTCGGCGC
GTCTGCGTCTGGCTGGCTGGCATAAATATCTCACTCGCAATCAAATTCAGCCGATAG
CGGAACGGGAAGGCGACTGGAGTGCCATGTCCGGTTTTCAACAAACCATGCAAATG
CTGAATGAGGGCATCGTTTCCACTGCGATGCTGGTTGCCAACGATCAGATGGCGCTG
GGCGCAATGCGCGCCATTACCGAGTCCGGGCTGCGCGTTGGTGCGGACATCTCGGTA
GTGGGATACGACGATACCGAAGACAGCTCATGTTATATCCCGCCGTTAACCACCATC
AAACAGGATTTTCGCCTGCTGGGGCAAACCAGCGTGGACCGCTTGCTGCAACTCTCT
CAGGGCCAGGCGGTTAAGGGCAATCAGCTGTTGCCCGTCTCACTGGTGAAAAGAAA
AACCACCCTGGCGCCCAATACGCAAACCGCCTCTCCCCGCGCGTTGGCCGATTCATT
AATGCAGCTGGCACGACAGGTTTCCCGACTGGAAAGCGGGCAGTGAGCGGTACCCG
ATAAAAGCGGCTTCCTGACAGGAGGCCGTTTTGTTTC (SEQ ID NO: 3)
MAKIDNAVLPEGSLVLVTGANGFVGSHVVEQLLEHGYKVRGTARSASKLANLQKRWD
AKYPGRFETAVVEDMLKQGAYDEVIKGAAGVAHIASVVSFSNKYDEVVTPAIGGTLNA
LRAAAATPSVKRFVLTSSTVSALIPKPNVEGIYLDEKSWNLESIDKAKTLPESDPQKSLW
VYAASKTEAELAAWKFMDENKPHFTLNAVNPNYTIGTIFDPETQSGSTSGWMMSLFNG
EVSPALALVPPQYYVSAVDIGLLHLGCLVLPQIERRRVYGTAGTFDWNTVLATFRKLYP
SKTFPADFPDQGQDLSKFDTAPSLEILKSLGRPGWRSIEESIKDLVGSETAGQTGHHHHH
H (SEQ ID NO: 4)
MAKIDNAVLPEGSLVLVTGANGFVGSHVVEQLLEHGYKVRGTARSASKLANLQKRWD
AKYPGRFETAVVEDMLKQGAYDEVIKGAAGVAHIASPVSFSNKYDEVVTPAIGGTLNAL
RAAAATPSVKRFVLTSSTMSALIPKPNVEGIYLDEKSWNLESIDKAKTLPESDPQKSLWV
YAASKTEAELAAWKFMDENKPHFTLNAVNPNYTIGTIFDPETQSGSTSGWMMSLENGE
VSPALALVPPQYYVSAVDIGLLHLGCLVLPQIERRRVYGTAGTFDWNTVLATFRKLYPS
KTFPADFPDQGQDLSKFDTAPSLEILKSLGRPGWRSIEESIKDLVGSETAGQTGHHHHHH
(SEQ ID NO: 5)
MKKIDNAVLPEGSLVLVTGANGFVGSHVVEQLLEHGYKVRGTARSASKLANLQKRWD
AKYPGRFETAVVEDMLKQGAYDEVIKGAAGVAHIASPVSFSNKYDEVVTPAIGGTLNAL
RAAAATPSVKRFVLTSSTMSALIPKPNVEGIYLDEKSWNLESIDKAKTLPESDPQKSLWV
YAASKTEAELAAWKFMDENKPHFTLNAVNPNYTIGTIFDPETQSGSTSGWMMSLENGE
VSPALALVPPQYYVSAVDIGLLHLGCLVLPQIERRRVYGTAGTFDWNTVLATFRKLYPS
KTFPADFPDQGQDLSKFDTAPSLEILKSLGRPGWRSIEESIKDLVGSETAGQTGHHHHHH
(SEQ ID NO: 6)
MKKIDNAVLPAGSLVLVTGANGFVGSHVVEQLLEHGYKVRGTARSASKLANLQKRWD
AKYPGRFETAVVEDMLKQGAYDEVIKGAAGVAHIASPVSFSNKYDEVVTPAIGGTLNAL
RAAAATPSVKRFVLTSSTMSALIPKPNVEGIYLDEKSWNLESIDKAKTLPESDPQKSLWV
YAASKTEAELAAWKFMDENKPHFTLNAVNPNYTIGTIFDPETQSGSTSGWMMSLENGE
VSPALALFRPQYYVSAVDIGLLHLGCLVLPQIERRRVYGTAGTFDWNTVLATFRKLYPS
KTFPADFPDQGQDLSKFDTAPSLEILKSLGRPGWRSIEESIKDLVGSETAGQTGHHHHHH
(SEQ ID NO: 7)
MKKIDNAVLPAGSLVLVTGANGFVGSHVVEQLLEHGYKVRGTARSASKLANLQKRWD
AKYPGRFETAVVEDMLKQGAYDEVIKGAAGVAHIASPVSFSNKYDEVVTPAIGGTLNAL
RAAAATPSVKRFVLTSSTMAALIPKPNVEGIYLDEKSWNLESIDKAKTLPESDPYKGLWV
YAASKTEAELLAWKFMDENKPHFTLNAVNPNYTIGTIFDPETQSGSTSGWMMKLENGE
VSPALALFRPQYYVSAVDIGLLHLGCLVLPQIERRRVYGTAGTFDWNTVLATFRKLYPS
KTFPADFPDQGQDLSKFDTAPSLEILKSLGRPGWRSFEESIKDLVGSETAGQTGHHHHHH
(SEQ ID NO: 8)
MKKIDNAVLPAGSLVLVTGANGFVGSHVVEQLLEHGYKVRGTARSASKLANLQKRWD
AKYPGRFETAVVEDMLKQGAYDEVIKGAAGVAHIASPVSFSNKYDEVVTPAIGGTLNAL
RAAAATPSVKRFVLTSSTMAALVPKPNVEGIYLDEKSWNLESIDKAKTLPESDPYKGLW
VYAASKTTAELLAWKFMDENKPHFTLNAVAPAYTIGTIFDPETQSGSTSGFMMKLENGE
VSPALALFRPQYYVSAVDIGLLHLGCLVLPQIERRRVYGTAGTFDWNTVLATFRKLYPS
KTFPADFPDQGQDLSKFDTAPSLEILKSLGRPGWRSFEESIKDLVGSETAGQTGHHHHHH
(SEQ ID NO: 9)
MKKIDNAVLPAGSLVLVTGANGFVGSHVVEQLLEHGYKVRGTARSASKLANLQKRWD
AKYPGRFETAVVEDMLKQGAYDEVIKGAAGVAHIASPVSFSNKYDEVVTPAIGGTLNAL
RAAAATPSVKRFVLTSSTMAALVPKPNVEGIYLDEKSWNLESIDKAKTLPESDPYKGLW
VYAASKTTAELLAWKFMDENKPHFTLNAVAPAYTIGTIFDPETQSGSTSGFMMKLENGE
VSPMLALFAPQYYVSAVDIGLLHLGCLVLPQIERRRVYGTAGTFDWNGVLATFRKLWPS
KTFPADFPDQGQDLSKFDTAPSLEILKSLGRPGWRSFEESIKDLVGSETAGQTGHHHHHH
(SEQ ID NO: 10)
MKKIDNAVLPAGSLVLVTGANGFVGSHVVEQLLEHGYKVRGTARSASKLANLQKRWD
AKYPGRFETAVVEDMLKQGAYDEVIKGAAGVAHIASPVSFSNKYDEVVTPAIGGTLNAL
RAAAATPSVKRFVLTSSTMAALVPKPNVEGIYLDEKSWNLESIDKAVTLPESDPEKGLW
VYAASKTTAELLAWKFMDENKPHFTLNAVAPAYTIGTIFDPETQSGSTSGFMMKLENGE
VSPMLALFGPQYYVSAVDIGLLHLGCLVLPQIERRRVYGTAGTFDWNGVLATFRKLWPS
KTFPADFPDQGQDLSKFDTAPSLEILKSLGRPGWRSFEDSIKDLVGSETAGQTGHHHHHH
(SEQ ID NO: 11)
MKKIDNAVLPAGSLVLVTGANGFVGSHVVEQLLEHGYKVRGTARSASKLANLQKRWD
AKYPGRFETAVVEDMLKQGAYDEVIKGAAGVAHIASPVLFSPKYDEVVTPAIGGTLNAL
RAAAATPSVKRFVLTSSTMAALVPKPNVEGIYLDEKSWNLESIDKAVTLPESDKEKGLW
VYAASKTTAEMLAWKFMDENKPHFTLNTVLPAYTIGTIFDPETQSGSTSGFMMKLENGE
VSPMLALFGPQYYVSAVDIGLLHLGCLVLPQIERRRVYGTAGTFDWNGVLATFRKLWPS
KTFPADFPDQGQDLSKFDTAPSLEILKSLGRPGWRSFEDSIKDLVGSETAGQTGHHHHHH
(SEQ ID NO: 12)
MKKIDNAVLPAGSLVLVTGANGFVGSHVVEQLLEHGYKVRGTARSASKLANLQKRWD
AKYPGRFETAVVEDMLKQGAYDEVIKGAAGVAHIASPVLFSPKYDEVVTPAIGGTLNAL
RAAAATPSVKRFVLTSSTMAALVPKPNVEGIYLDEKSWNLESIDKAVTLPESDKEKGLW
VYAASKTTAEMLAWKFMDENKPHFTLNTVLPAYTIGTIFDPETQSGSTSGFVMKLENGE
VSPMLALFGPQHYVSAVDIGLLHLGCLVLPQIERRRVYGTAGTFDWNGVLATFRKLWPS
KTFPADFPDQGQDLSKFDTTPSLEILKSLGRPGWRSFEDSIKDLVGSEFDGQTGHHHHHH
(SEQ ID NO: 13)
MKKIDNAVLPAGSLVLVTGANGFVGSHVVEQLLEHGYKVRGTARSASKLANLQKRWD
AKYPGRFETAVVEDMLKQGAYDEVIKGAAGVAHIASPVLFQPKYDEVVTPAIGGTLNA
LRAAAATPSVKRFVLTSSMMAAYVPKPNVQGIYLDEKSWNLESIDKAVTLPESDKEKGL
WVYAASKTMAEMLAWKFMDENKPHFTLNTVLPAYTIGTIFDPETQSGSTSGIVMKLFN
GEVSPMLALVGPQHYVSAVDIGLLHLGCLVLPQIERRRVYGTAGPFDWNMVLATFRKL
WPSKTFPADFPDQGQDLSVFDTRPSLEILKSLGRPGWRSFEDSIKDLVGSEFDGQTGHHH
HHH (SEQ ID NO: 14)
MKKIDNAVLPAGSLVLVTGANGFVGSHVVEQLLEHGYKVRGTARSASKLANLQKRWD
AKYPGRFETAVVEDMLKQGAYDEVIKGAAGVAHIASPVSFSPKYDEVVTPAIGGTLNAL
RAAAATPSVKRFVLTSSMMAAYVPKPNVQGIYLDEKSWNLESIDKAPTLPESHKEKGL
WVYAASKTMAEMLAWKFMDENKPHFTLNTVLPAYTIGTIFDPETQSGSTSGIVMKLFN
GEVSPMLALFGPQHYVSAVDIGLLHLGCLVLPQIERRRVYGTAGPFDWNMVLATFRKL
WPSKTFPADFPDQGQDLSVFDTRPSLEILKSLGREGWRSFEDSIKDLVGSEFDGQTGHHH
HHH (SEQ ID NO: 15)
MKKIDNAVLPAGSLVLVTGANGFVGSHVVEQLLEHGYKVRGTARSASKLANLQKRWD
AKYPGRFETAVVEDMLKDGAYDEVIKGAAGVAHIASPVSFSPKYDEVVPPAIGGTLNAL
RAAAATPSVKRFVLTSSMMAAIVPKPNVQGIYLDEKSWNLESIDKAVTLPESDKEKGLW
VYAASKTMAEMLAWKFMDENKPHFTLNTVLPAYTIGTIFDPETQSGSTSGIVMKLENGE
VSPMLALFGPQHYVSAFDIGLLHLGCLVLPQIERRRVYGTAGPFDWNMVLATFRKLWPS
KTFPADFPDQGQDLSVFDTRPSLEILKSLGRPGWRSFEDSIKDLVGSEFDGQTGHHHHHH
(SEQ ID NO: 16)
ATGGCTAAAATCGATAACGCAGTTCTGCCGGAAGGTTCCCTGGTTCTGGTTACCGGT
GCTAACGGTTTCGTTGGTTCCCACGTTGTTGAACAGCTGCTGGAACACGGTTACAAA
GTTCGTGGTACCGCTCGTTCCGCTTCCAAACTGGCTAACCTGCAGAAACGTTGGGAC
GCTAAATACCCGGGTCGTTTCGAAACCGCTGTTGTTGAAGACATGCTGAAACAGGGT
GCTTACGACGAAGTTATCAAAGGTGCTGCTGGTGTTGCTCACATCGCTTCCGTTGTTT
CCTTCTCCAACAAATACGACGAAGTTGTTACCCCGGCTATCGGTGGTACCTTGAACG
CTCTGCGTGCTGCTGCTGCTACCCCGTCCGTTAAACGTTTCGTTCTGACCTCCTCCAC
CGTTTCCGCTCTGATTCCGAAACCGAACGTTGAAGGTATCTACCTGGACGAAAAATC
CTGGAACCTGGAATCCATCGACAAAGCTAAAACCCTGCCGGAATCCGACCCGCAGA
AATCCCTGTGGGTATACGCTGCATCCAAGACCGAAGCTGAACTGGCTGCATGGAAAT
TTATGGATGAGAACAAGCCACACTTCACTCTGAACGCTGTACTGCCAAACTACACTA
TTGGCACTATTTTCGATCCGGAAACTCAGTCCGGTTCCACCTCCGGTTGGATGATGTC
CCTGTTTAACGGCGAGGTTTCCCCGGCTCTGGCTCTGATGCCACCGCAGTACTACGTT
TCCGCTGTTGATATTGGCCTGCTGCACCTGGGTTGCCTGGTTCTGCCACAAATCGAAC
GTCGTCGTGTTTACGGTACGGCTGGTACTTTCGATTGGAACACCGTTCTGGCTACCTT
CCGTAAACTGTACCCGTCCAAAACCTTCCCGGCTGACTTCCCAGATCAAGGTCAGGA
CCTGTCTAAATTCGACACCGCTCCGTCCCTGGAAATTCTGAAATCTCTGGGTCGCCCA
GGTTGGCGTTCCATCGAAGAATCCATCAAAGACCTGGTTGGTTCCGAAACCGCTGGC
CAAACTGGCCACCATCACCATCACCATTAG (SEQ ID NO: 17)

It will be appreciated that various of the above-discussed and other features and functions, or alternatives thereof, may be desirably combined into many other different systems or applications. Also that various presently unforeseen or unanticipated alternatives, modifications, variations, or improvements therein may be subsequently made by those skilled in the art which are also intended to be encompassed by the following claims.

1. A polypeptide comprising an amino acid sequence having at least 90% sequence identity to SEQ ID NO: 2.

2. The polypeptide according to claim 1, wherein the amino acid sequence has at least a 95% sequence identity to SEQ ID NO: 2.

3. The polypeptide according to claim 1, wherein the amino acid sequence has at least a 98% sequence identity to SEQ ID NO: 2.

4. The polypeptide according to claim 1, wherein the amino acid sequence consists of SEQ ID NO: 2.

5. The polypeptide according to claim 1, consisting of SEQ ID NO: 2.

6. The polypeptide according to claim 1, wherein at least at least one of the following conditions are satisfied:
(a) amino acid (aa) residue 2 of SEQ ID NO:2 is other than alanine, or
(b) aa residue 11 of SEQ ID NO:2 is other than glutamic acid.

7. A polynucleotide encoding the polypeptide according to claim 1.

8. The polynucleotide of claim 7, wherein the polynucleotide comprises SEQ ID NO:3.

9. The polynucleotide of claim 7, wherein at least one of the following conditions are satisfied:
(a) the triplet codon encoding the amino acid residue at position 2 of the polypeptide is other than GCT;
(b) the triplet codon encoding the amino acid residue at position 3 of the polypeptide is other than AAA;
(c) the triplet codon encoding the amino acid residue at position 4 of the polypeptide is other than ATC; or
(d) the triplet codon encoding the amino acid residue at position 11 of the polypeptide is other than GAA.

10. An expression vector comprising the polynucleotide according to claim 7, operably linked to one or more control sequences suitable for directing expression of the encoded polypeptide in a host cell.

11. The expression vector of claim 10, wherein the control sequence comprises a promoter.

12. The expression vector of claim 11, wherein the promoter comprises an E. coli promoter.

13. A host cell comprising the expression vector of claim 11.

14. The host cell of claim 12, wherein said host cell is E. coli.

15. A process for precipitating proteins from a cell lysate containing a polypeptide comprising an amino acid sequence having at least a 90% sequence identity to SEQ ID NO: 2, comprising treating the cell lysate containing the polypeptide to result in a cell lysate composition comprising >20% isopropanol.

16. The process of claim 15, wherein the volume percent of isopropanol in the cell lysate composition is about 25%.
